(19) United States
(12) Patent Application Publication
(10) Pub. No.: US 2026/0286391 A1
(43) Pub. Date: Sep. 24, 2026

(54) VECTOR PRODUCTION

(71) Applicants: OSPEDALE SAN RAFFAELE S.R.L., Milan (IT); FONDAZIONE TELETHON ETS, Rome (IT)

(72) Inventors: Alessio Cantore, Milan (IT); Michela Milani, Milan (IT); Chiara Simoni, Milan (IT); Cesare Canepari, Milan (IT)

(21) Appl. No.: 19/478,217

(22) PCT Filed: Apr. 29, 2024
(86) PCT No.: PCT/EP2024/061802

§ 371 Date: Oct. 23, 2025

(30) Foreign Application Priority Data
Apr. 28, 2023 (EP) 23170841.3

(51) Int. Cl. C12N 15/86; C12N 9/64

(52) U.S. Cl. CPC C12N 15/86; C12N 9/6454; C12Y 304/21061; C12N 2740/15023; C12N 2740/15043; C12N 2740/15052; C12N 2760/20222

(57) ABSTRACT

An enveloped viral particle producer or packaging cell, wherein the cell is modified to decrease expression of low density lipoprotein receptor (LDLR) on the surface of the cell.

### FIELD OF THE INVENTION

The present invention relates to cells and methods for producing enveloped viral particles. The invention also relates to cells and methods for producing enveloped virus-like particles (VLPs). In particular, the invention relates cells modified to decrease expression of low density lipoprotein receptor (LDLR) on the surface of the cell, and uses thereof.

### BACKGROUND TO THE INVENTION

Gene therapy involves the incorporation of genetic material into a cell to treat or prevent disease. The genetic material may supplement defective genes with functional copies of those genes, inactivate improperly functioning genes or introduce new therapeutic genes to a cell. Delivery of genetic material to a cell may be achieved through use of vectors which facilitate the transfer of nucleic acids. Viruses may be engineered to deliver a nucleotide of interest (NOI) to a target cell and are commonly employed as vectors in gene therapy. Viruses that have been used in gene therapy to date include lentiviruses, retroviruses, adenoviruses (AdV), adeno-associated viruses (AAV), herpes simplex viruses (HSV) and vaccinia viruses.

Lentiviral vectors (LV), such as those derived from HIV-1, represent an efficient and versatile platform for gene therapy. LV can transfer genes to both dividing and non-dividing cells, can stably integrate in the genome of target cells and have relatively large cargo capacity. In addition, there is low prevalence of pre-existing anti-vector immunity in humans. LVs are being used in ex vivo gene therapy and have shown promising results in pre-clinical studies involving direct in vivo administration, such as in liver-directed gene therapy.

However, there remains a significant need for improvements in viral vector production, for example to obtain high yield and high quality therapeutic products. One limiting factor may be the loss of infectious viral particles during the production process. For example, this may be due, at least in part, to re-infection of producer cells, which remain in contact with produced viruses (e.g. lentiviruses) during the production. This problem may become more acute when cells are grown in suspension at high density, a condition that is used in some cases for clinical grade production.

### SUMMARY OF THE INVENTION

The present inventors have demonstrated that decreased expression of low-density lipoprotein receptor (LDLR) on the surface of cells used to produce the enveloped viral particles leads to improvements in the production of the viral particles.

LDLR is a transmembrane protein that recognizes the apoprotein that builds LDL particles. When a LDL particle binds to the LDLR it is internalized into the cell and degraded while the LDLR recirculates to the cell surface. While not wishing to be bound by theory, modifying the cells to decrease expression of LDLR on the surface may interfere with the entry step during virus (e.g. lentivirus) infection.

The inventors increased the yield of lentivirus production by overexpressing proprotein convertase subtilisin kexin type 9 (PCSK9) in order to reduce the amount of LDLR on the surface of producer cells and consequently their reinfection during LV production. The inventors tested wild-type PCSK9 and the non-secreted PCSK9.S127R mutant during LV production, and evaluated the increase in titers and their transduction potential both in vitro, on T cells, and in vivo in mice, following intravenous (i.v.) administration.

The inventors have shown that amounts of physical viral particles and their infectivity are increased by using their modified cells. The inventors further observed that viruses produced using their modified cells comprised increased levels of VSV-G on the surface. Furthermore, the inventors have shown that the decreased LDLR expression surprisingly does not negatively impact the transduction efficiency of the viral particles that are produced, and even improves transduction efficiency and vector copy number in transduced cells in comparison to controls.

The inventors' approach may be applied to enveloped virus-like particles (VLPs) as well as enveloped viral particles.

In one aspect, the invention provides an enveloped viral particle producer or packaging cell, wherein the cell is modified to decrease expression of low density lipoprotein receptor (LDLR) on the surface of the cell.

In another aspect, the invention provides an enveloped viral particle packaging cell, wherein the cell is modified to decrease expression of low density lipoprotein receptor (LDLR) on the surface of the cell.

The cell may be, for example, modified (e.g. genetically engineered) to overexpress pro-protein convertase subtilisin/Kexin type 9 (PCSK9).

In some embodiments, the cell comprises a heterologous polynucleotide comprising a nucleotide sequence encoding pro-protein convertase subtilisin/Kexin type 9 (PCSK9).

In some embodiments, expression of endogenous pro-protein convertase subtilisin/Kexin type 9 (PCSK9) is increased. For example, endogenous PCSK9 expression may be increased using a transcription activator (e.g. an engineered transcription activator).

In another aspect, the invention provides a method of producing enveloped viral particles comprising the steps:
(a) introducing a transfer vector and optionally one or more helper vector into a cell;
(b) introducing a vector comprising a nucleotide sequence encoding pro-protein convertase subtilisin/Kexin type 9 (PCSK9) into the cell;
(c) culturing the cell under conditions suitable for the production of the enveloped viral particles.

In some embodiments, the steps (a), (b) and (c) are carried our consecutively in the order listed. In some embodiments, steps (a) and (b) are carried out at the same time. In some embodiments, step (b) is carried out before step (a), optionally to integrate the nucleotide sequence encoding PCSK9 into the genome of the cell.

The method may increase viral titer compared to production of the enveloped viral particles in the absence of the heterologous polynucleotide or vector comprising a nucleotide sequence encoding PCSK9. The method may increase amount of physical particles compared to production of the enveloped viral particles in the absence of the heterologous polynucleotide or vector comprising a nucleotide sequence encoding PCSK9.

Enveloped viral particles obtained by the method may exhibit increased transduction efficiency compared to enveloped viral particles produced in the absence of the heterologous polynucleotide or vector comprising a nucleotide sequence encoding PCSK9. Enveloped viral particles obtained by the method may exhibit increased infectivity compared to enveloped viral particles produced in the absence of the heterologous polynucleotide or vector comprising a nucleotide sequence encoding PCSK9.

In another aspect, the invention provides a method of producing enveloped virus-like particles (VLPs) comprising the steps:
(a) introducing a vector comprising a nucleotide sequence encoding pro-protein convertase subtilisin/Kexin type 9 (PCSK9) into an enveloped viral particle packaging cell; and
(b) culturing the cell under conditions suitable for the production of the enveloped VLPs.

In another aspect, the invention provides a method of producing enveloped virus-like particles (VLPs) comprising the steps:
(a) introducing one or more helper vector into a cell;
(b) introducing a vector comprising a nucleotide sequence encoding pro-protein convertase subtilisin/Kexin type 9 (PCSK9) into the cell; and
(b) culturing the cell under conditions suitable for the production of the enveloped VLPs.

In some embodiments, the steps (a), (b) and (c) are carried our consecutively in the order listed. In some embodiments, steps (a) and (b) are carried out at the same time. In some embodiments, step (b) is carried out before step (a), optionally to integrate the nucleotide sequence encoding PCSK9 into the genome of the cell.

The method may increase VLP titer compared to production of the enveloped VLPs in the absence of the heterologous polynucleotide or vector comprising a nucleotide sequence encoding PCSK9. The method may increase amount of physical VLPs compared to production of the enveloped VLPs in the absence of the heterologous polynucleotide or vector comprising a nucleotide sequence encoding PCSK9.

Enveloped VLPs obtained by the method may exhibit increased transduction efficiency compared to enveloped VLPs produced in the absence of the heterologous polynucleotide or vector comprising a nucleotide sequence encoding PCSK9. Enveloped VLPs obtained by the method may exhibit increased infectivity compared to enveloped VLPs produced in the absence of the heterologous polynucleotide or vector comprising a nucleotide sequence encoding PCSK9.

In another aspect, the invention provides an enveloped viral particle producer or packaging cell, wherein the cell comprises a heterologous polynucleotide comprising a nucleotide sequence encoding pro-protein convertase subtilisin/Kexin type 9 (PCSK9).

In another aspect, the invention provides an enveloped viral particle packaging cell, wherein the cell comprises a heterologous polynucleotide comprising a nucleotide sequence encoding pro-protein convertase subtilisin/Kexin type 9 (PCSK9).

In some embodiments, the cell overexpresses pro-protein convertase subtilisin/Kexin type 9 (PCSK9).

In some embodiments, the PCSK9 is transiently expressed (e.g. from the heterologous polynucleotide). The PCSK9 transient expression may be, for example, for less than about 96, 72, 48 or 24 hours, for example about 12-72 hours, 12-48 hours, 12-24 hours or 12-18 hours, such as about 14-16 hours.

In some embodiments, the PCSK9 is stably expressed (e.g. from the heterologous polynucleotide).

In some embodiments, the enveloped viral particle producer or packaging cell is modified (e.g. genetically engineered) to constitutively express PCSK9.

In some embodiments, the heterologous polynucleotide is a vector. In some embodiments, the heterologous polynucleotide is a plasmid.

In some embodiments, the heterologous polynucleotide is integrated into the genome of the cell.

In some embodiments, the PCSK9 is human PCSK9.

In some embodiments, the nucleotide sequence encoding PCSK9 comprises or consists of a nucleotide sequence that has at least 75%, 80%, 85%, 90%, 95%, 96%, 97%, 98% or 99% sequence identity to SEQ ID NO: 1. In some embodiments, the nucleotide sequence encoding PCSK9 comprises or consists of a nucleotide sequence that has at least 75%, 80%, 85%, 90%, 95%, 96%, 97%, 98% or 99% sequence identity to SEQ ID NO: 2.

In some embodiments, the nucleotide sequence encoding PCSK9 comprises or consists of the nucleotide sequence of SEQ ID NO: 1. In some embodiments, the nucleotide sequence encoding PCSK9 comprises or consists of the nucleotide sequence of SEQ ID NO: 2.

In some embodiments, the PCSK9 comprises or consists of an amino acid sequence that has at least 75%, 80%, 85%, 90%, 95%, 96%, 97%, 98% or 99% sequence identity to SEQ ID NO: 3. In some embodiments, the PCSK9 comprises or consists of an amino acid sequence that has at least 75%, 80%, 85%, 90%, 95%, 96%, 97%, 98% or 99% sequence identity to SEQ ID NO: 4.

In some embodiments, the PCSK9 comprises or consists of the amino acid sequence of SEQ ID NO: 3. In some embodiments, the PCSK9 comprises or consists of the amino acid sequence of SEQ ID NO: 4.

In preferred embodiments, the PCSK9 comprises the mutation S127R, wherein the amino acids are numbered with reference to SEQ ID NO: 4.

In some embodiments, the cell is a HEK-293 cell or a derivative thereof. In some embodiments, the cell is a HEK-293T or a HEK-293 T-REx cell. In some embodiments, the cell is a HEK-293T cell.

In some embodiments, the enveloped viral particle is a lentivirus, retrovirus, herpes simplex virus, vaccinia virus, hepadnavirus, togavirus, flavivirus, arenavirus, coronavirus, orthomyxovirus, paramyxovirus, bunyavirus, bornavirus, rhabdovirus or filovirus, or is derived therefrom.

In preferred embodiments, the enveloped viral particle is a lentivirus.

In preferred embodiments, the enveloped viral particle is vesicular stomatitis virus glycoprotein G (VSV-G) pseudotyped. In preferred embodiments, the enveloped viral particle is a VSV-G pseudotyped lentivirus.

In some embodiments, the lentivirus is a self-inactivating lentivirus.

In some embodiments, the enveloped VLP is a lentivirus VLP, retrovirus VLP, herpes simplex virus VLP, vaccinia virus VLP, hepadnavirus VLP, togavirus VLP, flavivirus VLP, arenavirus VLP, coronavirus VLP, orthomyxovirus VLP, paramyxovirus VLP, bunyavirus VLP, bornavirus VLP, rhabdovirus VLP or filovirus VLP, or is derived therefrom.

In preferred embodiments, the enveloped VLP is a lentivirus VLP.

In preferred embodiments, the enveloped VLP is vesicular stomatitis virus glycoprotein G (VSV-G) pseudotyped. In preferred embodiments, the enveloped VLP is a VSV-G pseudotyped lentivirus VLP.

In some embodiments, the enveloped viral particle comprises a polynucleotide comprising a transgene. In some embodiments, the transgene is a therapeutic transgene. In some embodiments, the transgene encodes a chimeric antigen receptor (CAR). In some embodiments, the transgene encodes Factor IX (FIX).

In some embodiments, the enveloped VLP comprises one or more component of gene editing machinery (e.g. CRISPR/Cas9, base editor or prime editor).

In another aspect, the invention provides a method of producing an enveloped viral particle producer or packaging cell, wherein the cell is modified to decrease expression of low density lipoprotein receptor (LDLR) on the surface of the cell, wherein the method comprises the step of introducing a polynucleotide comprising a nucleotide sequence encoding pro-protein convertase subtilisin/Kexin type 9 (PCSK9) into the cell. In another aspect, the invention provides a method of producing an enveloped viral particle packaging cell, wherein the cell is modified to decrease expression of low density lipoprotein receptor (LDLR) on the surface of the cell, wherein the method comprises the step of introducing a polynucleotide comprising a nucleotide sequence encoding pro-protein convertase subtilisin/Kexin type 9 (PCSK9) into the cell. The introducing may be, for example, by transfection or transduction. The introducing may be, for example, transient or by integration of the nucleotide sequence encoding PCSK9 into the genome of the cell.

In another aspect, the invention provides use of the enveloped viral particle producer cell according to the invention for the production of enveloped viral particles.

In another aspect, the invention provides use of the enveloped viral particle packaging cell of the invention for the production of enveloped virus-like particles (VLPs).

In another aspect, the invention provides a method of producing enveloped viral particles comprising the steps of:
(a) providing an enveloped viral particle producer cell according to the invention; and
(b) culturing the cell under conditions suitable for the production of enveloped viral particles.

In another aspect, the invention provides a method of producing enveloped virus-like particles (VLPs) comprising the steps of:
(a) providing an enveloped viral particle packaging cell according to the invention; and
(b) culturing the cell under conditions suitable for the production of enveloped VLPs.

In some embodiments, the cell is cultured in suspension. In some embodiments, the culturing is in a bioreactor.

In another aspect, the invention provides an enveloped viral particle produced by the method of the invention.

In another aspect, the invention provides an enveloped virus-like particle (VLP) produced by the method of the invention.

In another aspect, the invention provides a cell transduced by the enveloped viral particle of the invention.

In another aspect, the invention provides a cell transduced by the enveloped virus-like particle (VLP) of the invention.

In some embodiments, the cell is a T cell. In some embodiments, the cell is a haematopoietic stem and/or progenitor cell (HSPC).

In another aspect, the invention provides a pharmaceutical composition comprising the enveloped viral particle of the invention or the cell of the invention, and a pharmaceutically acceptable carrier, diluent or excipient.

In another aspect, the invention provides a pharmaceutical composition comprising the enveloped virus-like particle (VLP) of the invention, and a pharmaceutically acceptable carrier, diluent or excipient.

In another aspect, the invention provides an enveloped viral particle of the invention for use in therapy. In another aspect, the invention provides an enveloped virus-like particle (VLP) of the invention for use in therapy. In another aspect, the invention provides a cell of the invention for use in therapy.

In some embodiments, the therapy comprises administering the enveloped viral particle to a subject in need thereof. In some embodiments, the therapy comprises administering the enveloped VLP to a subject in need thereof.

In another aspect, the invention provides use of an enveloped viral particle of the invention for the manufacture of a medicament. In another aspect, the invention provides use of an enveloped virus-like particle (VLP) of the invention for the manufacture of a medicament. In another aspect, the invention provides use of a cell of the invention for the manufacture of a medicament.

In another aspect, the invention provides a method of gene therapy comprising administering the enveloped viral particle of the invention to a subject in need thereof. In another aspect, the invention provides a method of gene therapy comprising administering the enveloped virus-like particle (VLP) of the invention to a subject in need thereof. In another aspect, the invention provides a method of gene therapy comprising administering the cell of the invention to a subject in need thereof.

In some embodiments, the enveloped viral particle is administered to a subject systemically.

In some embodiments, the enveloped VLP is administered to a subject systemically.

In some embodiments, the enveloped viral particle is administered to a subject by intravenous injection. In some embodiments, the enveloped VLP is administered to a subject by intravenous injection.

### DESCRIPTION OF THE DRAWINGS

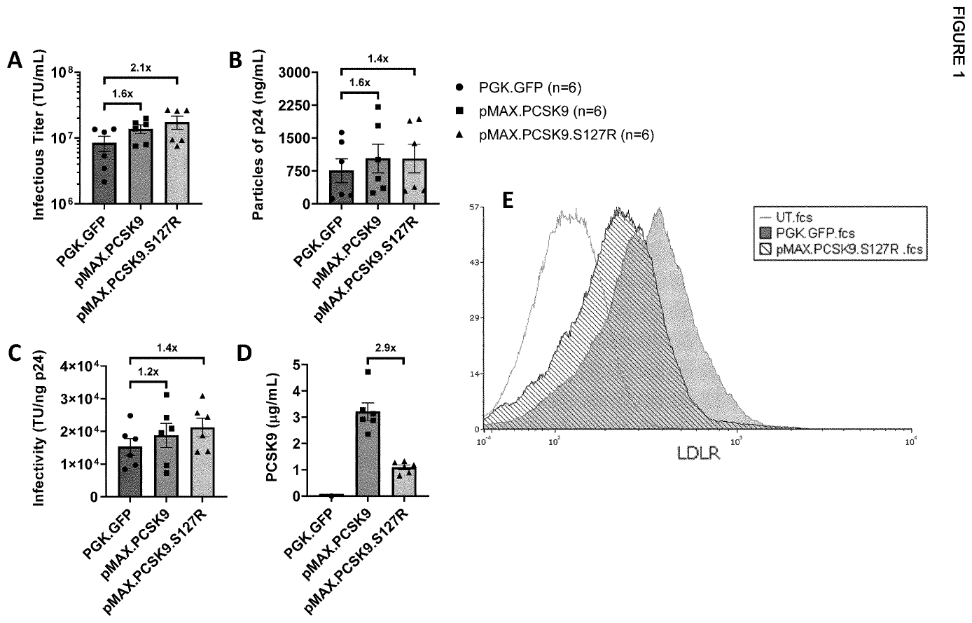

### FIG. 1

LV Production with Overexpression of PCSK9.

A-C. Mean with standard error of the mean (SEM) and single values of infectious titer (TU/mL, n=6, A), physical particles (pg HIV Gag p24/mL, n=6, B), or specific infectivity (TU/ng p24, n=6, C) of control PGK.GFP LV (circles), or LV produced with the addition of pMAX.coPCSK9 (square), or pMAX.coPCSK9.S127R (triangle) to the transfection mix. Pool of 2 independent experiments. D. Mean with SEM and single values of PCSK9 concentration (μg/mL) measured by ELISA in the LV-containing supernatant after production LV shown in (A-C). Pool of 2 independent experiments E. Representative histograms of flow cytometry analysis of 293T cells left unstained (gray line), or stained with anti-LDLR antibody after production of control PGK.GFP LV (filled gray line) or LV produced with the addition of pMAX.coPCSK9.S127R (filled black line) to the transfection mix analyzed 54 h after transfection.

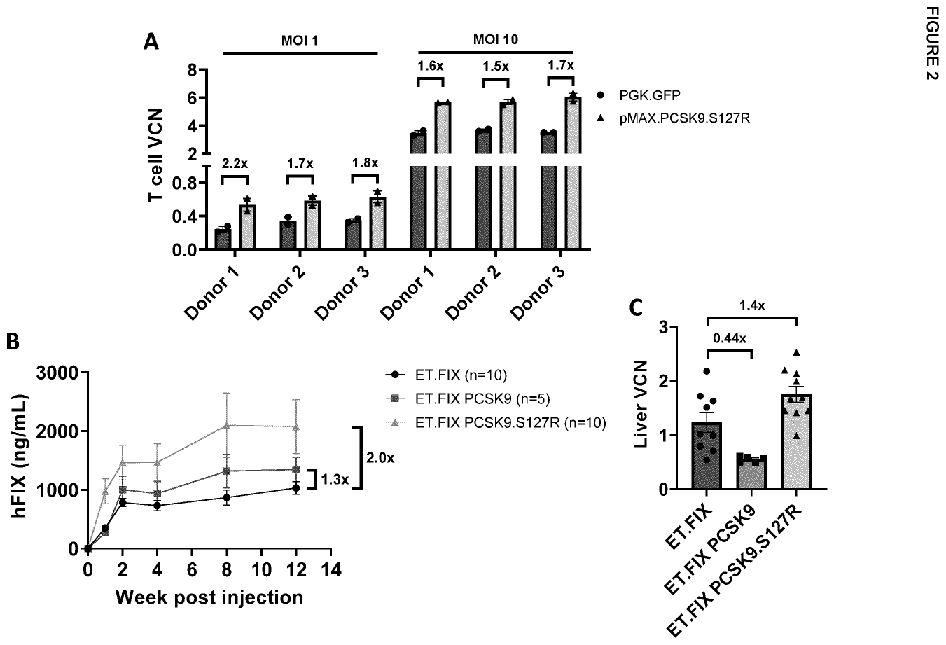

### FIG. 2

In vitro and in vivo evaluation of transduction efficiency of LV produced with PCSK9 overexpression.

A. Mean with SEM and single values of vector copy number (VCN) measured in T cells transduced at multiplicity of infection (MOI) 1 or 10 with control PGK.GFP LV (circle) or with LV produced with the addition of pMAX.coPCSK9.S127R (triangles) to the transfection mix, analyzed 10 days after transduction (n=2 technical replicates, n=3 healthy donors). B. Mean with SEM of human factor IX (hFIX) concentration in the plasma of mice treated with control ET.FIX LV (circles, n=10), or with LV produced with the addition of pMAX.coPCSK9 (square, n=5) or pMAX.coPCSK9.S127R (triangle, n=10) to the transfection mix, analyzed at the indicated time point. Pool of 2 independent experiments. C. Mean with SEM and single values of VCN measured in the liver of mice shown in (B) 12 weeks after LV administration. Pool of 2 independent experiments.

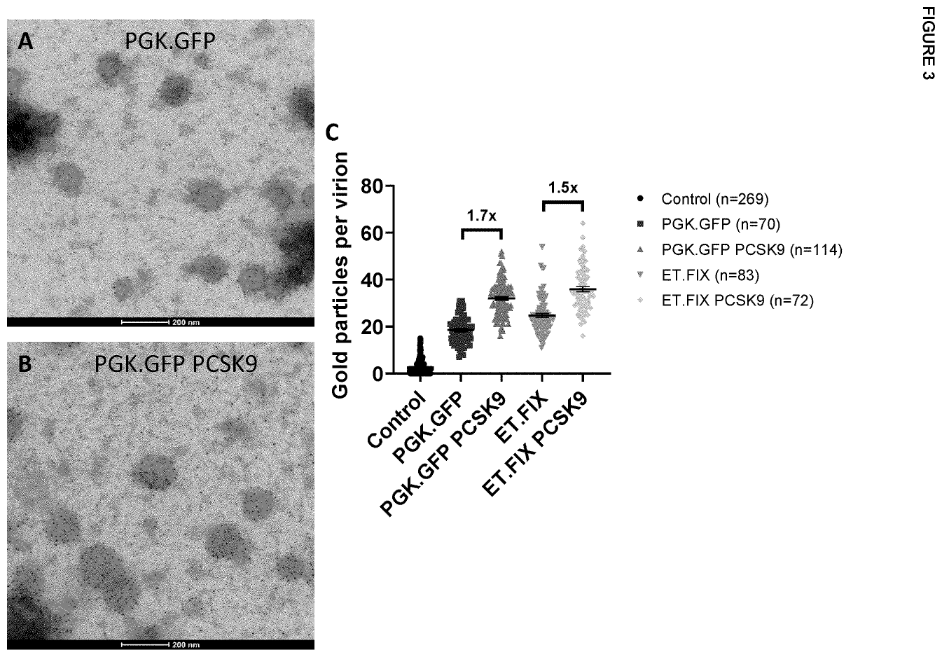

### FIG. 3

### Quantification of VSV.G Content on LV Envelope.

A, B. Representative images of control PGK.GFP LV (A) or LV produced by adding pMAX.coPCSK9.S127R to the transection mix (B) immunostained with anti-VSV.G antibody and analyzed by transmission electron microscopy. Scale bars: 200 nm. C. Mean with SEM and single values of gold particles per virion immunostained with anti-VSV.G antibody counted on control PGK.GFP LV (square, n=70), PGK.GFP LV produced with pMAX.coPCSK9.S127R (triangle, n=114), control ET.FIX LV (inverted triangles, n=83), ET.FIX LV produced with pMAX.coPCSK9.S127R (rhombus, n=72), or without the primary antibody (control, circles, n=269), and analyzed by electron microscopy.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

### Low Density Lipoprotein Receptor (LDLR)

In one aspect, the invention provides an enveloped viral particle producer or packaging cell, wherein the cell is modified to decrease expression of low density lipoprotein receptor (LDLR) on the surface of the cell.

The low density lipoprotein (LDL) receptor (LDLR or LDL-R) is a protein that mediates the endocytosis of cholesterol-rich LDL. LDLR binds LDL, which is the major cholesterol-carrying lipoprotein of plasma, and transports it into cells by endocytosis.

The LDLR may be, for example, human LDLR.

An example LDLR amino acid sequence is:

(SEQ ID NO: 5; human LDLR)
MGPWGWKLRWTVALLLAAAGTAVGDRCERNEFQCQDGKCISYKWVCDGSAECQDGSDESQETCLSVTC
KSGDFSCGGRVNRCIPQFWRCDGQVDCDNGSDEQGCPPKTCSQDEFRCHDGKCISRQFVCDSDRDCLD
GSDEASCPVLTCGPASFQCNSSTCIPQLWACDNDPDCEDGSDEWPQRCRGLYVFQGDSSPCSAFEFHC
LSGECIHSSWRCDGGPDCKDKSDEENCAVATCRPDEFQCSDGNCIHGSRQCDREYDCKDMSDEVGCVN
VTLCEGPNKFKCHSGECITLDKVCNMARDCRDWSDEPIKECGTNECLDNNGGCSHVCNDLKIGYECLC
PDGFQLVAQRRCEDIDECQDPDTCSQLCVNLEGGYKCQCEEGFQLDPHTKACKAVGSIAYLEFTNRHE
VRKMTLDRSEYTSLIPNLRNVVALDTEVASNRIYWSDLSQRMICSTQLDRAHGVSSYDTVISRDIQAP
DGLAVDWIHSNIYWTDSVLGTVSVADTKGVKRKTLFRENGSKPRAIVVDPVHGFMYWTDWGTPAKIKK
GGLNGVDIYSLVTENIQWPNGITLDLLSGRLYWVDSKLHSISSIDVNGGNRKTILEDEKRLAHPESLA
VFEDKVEWTDIINEAIFSANRLTGSDVNLLAENLLSPEDMVLFHNLTQPRGVNWCERTTLSNGGCQYL
CLPAPQINPHSPKFTCACPDGMLLARDMRSCLTEAEAAVATQETSTVRLKVSSTAVRTQHTTTRPVPD
TSRLPGATPGLTTVEIVTMSHQALGDVAGRGNEKKPSSVRALSIVLPIVLLVFLCLGVELLWKNWRLK
NINSINFDNPVYQKTTEDEVHICHNQDGYSYPSRQMVSLEDDVA

In some embodiments, the LDLR comprises or consists of an amino acid sequence that has at least 75%, 80%, 85%, 90%, 95%, 96%, 97%, 98% or 99% sequence identity to SEQ ID NO: 5, or a fragment thereof.

In some embodiments, the LDLR comprises or consists of the amino acid sequence of SEQ ID NO: 5, or a fragment thereof.

A fragment and/or variant of LDLR may retain LDLR activity (e.g. the activity of SEQ ID NO: 5). For example, a fragment and/or variant of LDLR may bind LDL and transport it into cells by endocytosis. Suitably, a fragment and/or variant of LDLR may have the same or similar activity to LDLR, for example may have at least 40%, at least 50%, at least 60%, at least 70%, at least 80%, at least 90%, at least 95% or 100% of the activity of LDLR (e.g. the LDLR of SEQ ID NO: 5). The skilled person will be able to generate fragments and/or variants, for example using conservative substitutions, based on the known structural and functional features of LDLR.

Decreased expression of LDLR on the surface of the cell refers to a decrease in the number of LDLR molecules that are expressed on the surface of the cell that has been modified (e.g. genetically engineered), in comparison to the number of LDLR molecules that are expressed on the surface of a cell lacking the modification, but under otherwise substantially identical conditions.

The expression of LDLR on the surface of the cell may be decreased such that the number of surface-exposed LDLR molecules is, for example, less than about 75%, 70%, 60%, 50%, 40%, 30%, 25%, 20%, 15%, 10%, 5%, 4%, 3%, 2% or 1% of the number of surface-exposed LDLR molecules that are displayed in the absence of the modification. Expression of LDLR on the surface of a cell may be readily determined by the skilled person using any suitable method known in the art, for example flow cytometry (e.g. as described herein).

In addition to decreased LDLR expression, expression of one or more other LDLR family members may also be decreased. LDLR family members may include VLDL receptor (VLDLR), ApoER2 (also known as LRP8), low density lipoprotein receptor-related protein 4 (also known as multiple epidermal growth factor (EGF) repeat-containing protein (MEGF7)), LDLR-related protein 1, LDLR-related protein 1b and Megalin.

Decreased expression of an LDLR family member on the surface of the cell refers to a decrease in the number of the LDLR family member molecules that are expressed on the surface of the cell that has been modified (e.g. genetically engineered), in comparison to the number of the LDLR family member molecules that are expressed on the surface of a cell lacking the modification, but under otherwise substantially identical conditions.

The expression of the LDLR family member on the surface of the cell may be decreased such that the number of surface-exposed molecules of the LDLR family member is, for example, less than about 75%, 70%, 60%, 50%, 40%, 30%, 25%, 20%, 15%, 10%, 5%, 4%, 3%, 2% or 1% of the number of surface-exposed molecules of the LDLR family member that are displayed in the absence of the modification. Expression of the LDLR family member on the surface of a cell may be readily determined by the skilled person using any suitable method known in the art, for example flow cytometry.

### Proprotein Convertase Subtilisin Kexin Type 9 (PCSK9)

The producer or packaging cell may be, for example, modified to overexpress pro-protein convertase subtilisin/Kexin type 9 (PCSK9). In some embodiments, the cell comprises a heterologous polynucleotide comprising a nucleotide sequence encoding pro-protein convertase subtilisin/Kexin type 9 (PCSK9).

An example PCSK9 amino acid sequence is:

(SEQ ID NO: 4)
MGTVSSRRSWWPLPLLLLLLLLLGPAGARAQEDEDGDYEELVLALRSEEDGLAEAPEHGTTATFHRCA
KDPWRLPGTYVVVLKEETHLSQSERTARRLQAQAARRGYLTKILHVFHGLLPGELVKMSGDLLELALK
LPHVDYIEEDSSVFAQSIPWNLERITPPRYRADEYQPPDGGSLVEVYLLDTSIQSDHREIEGRVMVTD
FENVPEEDGTRFHRQASKCDSHGTHLAGVVSGRDAGVAKGASMRSLRVLNCQGKGTVSGTLIGLEFIR
KSQLVQPVGPLVVLLPLAGGYSRVLNAACQRLARAGVVLVTAAGNERDDACLYSPASAPEVITVGATN
AQDQPVTLGTLGTNFGRCVDLFAPGEDIIGASSDCSTCFVSQSGTSQAAAHVAGIAAMMLSAEPELTL
AELRQRLIHFSAKDVINEAWFPEDQRVLTPNLVAALPPSTHGAGWQLFCRTVWSAHSGPTRMATAVAR
CAPDEELLSCSSFSRSGKRRGERMEAQGGKLVCRAHNAFGGEGVYAIARCCLLPQANCSVHTAPPAEA
SMGTRVHCHQQGHVLTGCSSHWEVEDLGTHKPPVLRPRGQPNQCVGHREASIHASCCHAPGLECKVKE
HGIPAPQEQVTVACEEGWTLTGCSALPGTSHVLGAYAVDNTCVVRSRDVSTTGSTSEGAVTAVAICCR
SRHLAQASQELQ

In some embodiments, the PCSK9 comprises or consists of an amino acid sequence that has at least 75%, 80%, 85%, 90%, 95%, 96%, 97%, 98% or 99% sequence identity to SEQ ID NO: 4, or a fragment thereof.

In some embodiments, the PCSK9 comprises or consists of the amino acid sequence of SEQ ID NO: 4, or a fragment thereof.

In preferred embodiments, the PCSK9 comprises the mutation S127R, wherein the amino acids are numbered with reference to SEQ ID NO: 4.

The terms “corresponding to”, “reference to” and “relative to” when used in the context of the numbering of a given amino acid or polynucleotide sequence may refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of a PCSK9, can be aligned to a reference sequence by introducing gaps to optimise residue matches between the two sequences. In these cases, although the gaps are present, the numbering of the residue in the given amino acid or polynucleotide sequence is made with respect to the reference sequence to which it has been aligned.

An example PCSK9 S127R amino acid sequence is:

(SEQ ID NO: 3)
MGTVSSRRSWWPLPLLLLLLLLLGPAGARAQEDEDGDYEELVLALRSEEDGLAEAPEHGTTATFHRCA
KDPWRLPGTYVVVLKEETHLSQSERTARRLQAQAARRGYLTKILHVFHGLLPGFLVKMRGDLLELALK
LPHVDYIEEDSSVFAQSIPWNLERITPPRYRADEYQPPDGGSLVEVYLLDTSIQSDHREIEGRVMVTD
FENVPEEDGTRFHRQASKCDSHGTHLAGVVSGRDAGVAKGASMRSLRVLNCQGKGTVSGTLIGLEFIR
KSQLVQPVGPLVVLLPLAGGYSRVLNAACQRLARAGVVLVTAAGNERDDACLYSPASAPEVITVGATN
AQDQPVTLGTLGTNFGRCVDLFAPGEDIIGASSDCSTCFVSQSGTSQAAAHVAGIAAMMLSAEPELTL
AELRQRLIHFSAKDVINEAWFPEDQRVLTPNLVAALPPSTHGAGWQLFCRTVWSAHSGPTRMATAVAR
CAPDEELLSCSSFSRSGKRRGERMEAQGGKLVCRAHNAFGGEGVYAIARCCLLPQANCSVHTAPPAEA
SMGTRVHCHQQGHVLTGCSSHWEVEDLGTHKPPVLRPRGQPNQCVGHREASIHASCCHAPGLECKVKE
HGIPAPQEQVTVACEEGWTLTGCSALPGTSHVLGAYAVDNTCVVRSRDVSTTGSTSEGAVTAVAICCR
SRHLAQASQELQ

In some embodiments, the PCSK9 comprises or consists of an amino acid sequence that has at least 75%, 80%, 85%, 90%, 95%, 96%, 97%, 98% or 99% sequence identity to SEQ ID NO: 3, or a fragment thereof. Preferably, the PCSK9 comprises the mutation S127R, wherein the amino acids are numbered with reference to SEQ ID NO: 4.

In some embodiments, the PCSK9 comprises or consists of the amino acid sequence of SEQ ID NO: 3, or a fragment thereof.

### Example Nucleotide Sequences Encoding PCSK9 Include:

(SEQ ID NO: 2; codon-optimised PCSK9)
ATGGGCACCGTGTCCAGCAGACGGTCTTGGTGGCCTCTGCCTCTGCTGTTGCTGCTGCTTCTTCTGCT
TGGACCTGCTGGCGCTAGAGCCCAAGAGGATGAGGATGGCGACTACGAGGAACTGGTGCTGGCCCTGA
GAAGCGAGGAAGATGGACTGGCCGAAGCTCCTGAGCACGGCACCACAGCCACCTTCCACAGATGCGCC
AAAGATCCTTGGAGACTGCCCGGCACATACGTGGTGGTGCTGAAAGAGGAAACCCACCTGAGCCAGAG
CGAGAGAACAGCCAGAAGGCTGCAGGCTCAGGCCGCCAGAAGAGGCTACCTGACCAAGATCCTGCACG
TGTTCCACGGCCTGCTGCCTGGCTTTCTGGTCAAGATGTCTGGCGATCTGCTGGAACTGGCTCTGAAG
CTGCCTCACGTGGACTACATCGAAGAGGACAGCAGCGTGTTCGCCCAGAGCATCCCCTGGAACCTGGA
AAGAATCACCCCTCCTAGATACCGGGCCGACGAGTACCAACCTCCTGATGGCGGATCTCTGGTGGAAG
TGTACCTGCTGGATACCAGCATCCAGAGCGACCACCGGGAAATCGAGGGCAGAGTGATGGTCACCGAC
TTCGAGAACGTGCCCGAAGAAGATGGCACCCGGTTTCACAGACAGGCCAGCAAGTGTGATAGCCACGG
AACACATCTGGCCGGCGTGGTGTCTGGAAGAGATGCTGGTGTTGCCAAGGGCGCCAGCATGAGATCTC
TGAGAGTGCTGAACTGCCAAGGCAAGGGCACAGTGTCTGGCACACTGATCGGCCTCGAGTTCATCCGG
AAGTCCCAGCTGGTTCAGCCTGTGGGACCTCTGGTTGTTCTGCTGCCACTGGCTGGCGGCTATAGCAG
GGTTCTGAATGCCGCCTGTCAGAGACTGGCTAGAGCTGGCGTTGTGCTGGTTACAGCCGCCGGAAACT
TCAGAGATGACGCCTGCCTGTACAGCCCTGCCAGTGCTCCTGAAGTGATCACAGTGGGCGCCACAAAC
GCCCAGGATCAGCCTGTTACACTGGGCACCCTGGGCACAAACTTCGGCAGATGCGTGGACCTGTTTGC
CCCTGGCGAGGATATTATCGGCGCCAGCTCCGATTGCAGCACCTGTTTTGTGTCTCAGAGCGGCACCT
CTCAGGCTGCCGCTCATGTTGCTGGAATCGCCGCCATGATGCTGTCTGCCGAGCCTGAACTGACTCTG
GCCGAGCTGAGACAGCGGCTGATCCACTTTAGCGCCAAGGACGTGATCAACGAGGCCTGGTTTCCCGA
GGATCAGAGGGTGCTGACCCCTAATCTGGTGGCTGCTCTGCCACCTTCTACACACGGTGCTGGCTGGC
AGCTGTTCTGCAGGACAGTTTGGAGCGCCCACAGCGGCCCTACAAGAATGGCTACAGCCGTGGCTAGA
TGCGCCCCTGATGAGGAACTGCTGAGCTGCTCCAGCTTCAGCAGAAGCGGCAAGAGAAGAGGCGAGCG
GATGGAAGCCCAAGGCGGAAAACTTGTGTGCAGAGCCCACAATGCCTTTGGCGGAGAAGGCGTGTACG
CCATTGCCAGATGTTGTCTGTTGCCCCAGGCCAACTGCAGCGTGCACACAGCTCCTCCAGCCGAAGCC
TCTATGGGCACCAGAGTGCACTGTCACCAGCAGGGACATGTGCTGACAGGCTGTAGCAGCCACTGGGA
AGTTGAGGACCTGGGAACCCACAAGCCTCCAGTGCTCAGACCTAGAGGCCAGCCTAATCAGTGCGTGG
GACACAGAGAGGCCTCCATCCACGCCTCTTGTTGTCATGCCCCTGGACTGGAATGCAAAGTGAAAGAG
CACGGAATCCCCGCTCCTCAAGAGCAAGTGACCGTGGCCTGTGAAGAAGGCTGGACACTGACCGGATG
TTCTGCCCTGCCTGGCACATCTCATGTGCTGGGAGCCTACGCCGTGGACAATACCTGTGTTGTGCGGA
GCAGAGATGTGTCCACCACCGGCTCTACATCTGAGGGCGCTGTGACAGCTGTGGCCATCTGCTGCAGA
AGCAGACACCTGGCACAGGCCTCTCAAGAGCTGCAGTGA
(SEQ ID NO: 1; codon-optimised PCSK9 S127R)
ATGGGCACCGTGAGCTCCCGGAGAAGCTGGTGGCCTCTGCCACTGTTATTACTGCTGCTGCTGCTGCT
GGGACCAGCAGGAGCAAGGGCCCAGGAGGACGAGGATGGCGACTACGAGGAGCTGGTGCTGGCCCTGC
GCTCCGAGGAGGACGGCCTGGCCGAGGCCCCTGAGCACGGCACCACAGCCACCTTCCACAGGTGCGCA
AAGGACCCCTGGAGGCTGCCAGGCACATACGTGGTGGTGCTGAAGGAGGAGACACACCTGTCCCAGTC
TGAGAGGACCGCAAGGCGCCTGCAGGCACAGGCAGCAAGGAGAGGCTATCTGACCAAGATCCTGCACG
TGTTCCACGGCCTGCTGCCAGGCTTTCTGGTGAAGATGAGGGGCGACCTGCTGGAGCTGGCCCTGAAG
CTGCCACACGTGGATTACATCGAGGAGGACTCTAGCGTGTTTGCCCAGTCTATCCCCTGGAACCTGGA
GAGAATCACCCCCCCTCGGTACAGAGCCGATGAGTATCAGCCACCAGACGGAGGCTCCCTGGTGGAGG
TGTATCTGCTGGATACAAGCATCCAGTCCGACCACCGGGAGATCGAGGGCAGAGTGATGGTGACAGAC
TTCGAGAACGTGCCTGAGGAGGATGGCACCAGGTTTCACCGCCAGGCCTCTAAGTGCGACAGCCACGG
CACCCACCTGGCAGGAGTGGTGAGCGGCCGGGATGCAGGAGTGGCAAAGGGAGCATCTATGCGGAGCC
TGAGAGTGCTGAATTGTCAGGGCAAGGGCACAGTGTCCGGCACCCTGATCGGCCTGGAGTTCATCCGG
AAGTCTCAGCTGGTGCAGCCAGTGGGACCACTGGTGGTGCTGCTGCCACTGGCAGGAGGATACAGCAG
AGTGCTGAACGCAGCATGCCAGAGGCTGGCAAGGGCAGGCGTGGTGCTGGTGACAGCCGCCGGCAACT
TCCGGGACGATGCCTGTCTGTATTCCCCCGCCTCTGCCCCTGAGGTGATCACAGTGGGAGCAACCAAC
GCACAGGACCAGCCTGTGACCCTGGGCACACTGGGCACCAATTTCGGCCGCTGCGTGGATCTGTTTGC
ACCAGGAGAGGACATCATCGGAGCATCCTCTGATTGCAGCACATGTTTCGTGAGCCAGTCCGGCACCT
CCCAGGCTGCCGCCCACGTGGCAGGAATCGCAGCAATGATGCTGAGCGCCGAGCCAGAGCTGACCCTG
GCAGAGCTGAGGCAGCGCCTGATCCACTTCTCCGCCAAGGACGTGATCAACGAGGCCTGGTTTCCTGA
GGATCAGAGGGTGCTGACACCAAATCTGGTGGCCGCCCTGCCTCCAAGCACCCACGGAGCCGGCTGGC
AGCTGTTTTGTAGAACAGTGTGGAGCGCCCACTCCGGACCAACAAGGATGGCAACCGCAGTGGCAAGA
TGCGCACCTGACGAGGAGCTGCTGTCCTGTAGCTCCTTCTCTAGGAGCGGAAAGAGGAGGGGAGAGAG
GATGGAGGCACAGGGAGGAAAGCTGGTGTGCAGGGCACACAACGCCTTTGGCGGAGAGGGCGTGTACG
CAATCGCAAGATGCTGTCTGCTGCCTCAGGCCAATTGTTCTGTGCACACAGCACCACCTGCAGAGGCA
AGCATGGGAACCAGGGTGCACTGCCACCAGCAGGGACACGTGCTGACCGGCTGTTCTAGCCACTGGGA
GGTGGAGGATCTGGGAACACACAAGCCACCCGTGCTGCGGCCAAGAGGACAGCCAAACCAGTGCGTGG
GCCACAGAGAGGCCTCCATCCACGCCTCTTGCTGTCACGCCCCAGGCCTGGAGTGTAAGGTGAAGGAG
CACGGCATCCCCGCACCTCAGGAGCAGGTGACCGTGGCATGCGAGGAGGGATGGACCCTGACAGGATG
TTCTGCCCTGCCAGGCACCAGCCACGTGCTGGGAGCATATGCAGTGGACAATACATGCGTGGTGAGGA
GCCGCGACGTGAGCACCACAGGCTCCACATCTGAGGGAGCAGTGACCGCAGTGGCAATCTGCTGTCGG
TCCAGACACCTGGCCCAGGCCTCTCAGGAGCTGCAGTGA

In some embodiments, the nucleotide sequence encoding PCSK9 comprises or consists of a nucleotide sequence that has at least 75%, 80%, 85%, 90%, 95%, 96%, 97%, 98% or 99% sequence identity to SEQ ID NO: 2, or a fragment thereof.

In some embodiments, the nucleotide sequence encoding PCSK9 comprises or consists of the nucleotide sequence of SEQ ID NO: 2, or a fragment thereof.

In some embodiments, the nucleotide sequence encoding PCSK9 comprises or consists of a nucleotide sequence that has at least 75%, 80%, 85%, 90%, 95%, 96%, 97%, 98% or 99% sequence identity to SEQ ID NO: 1, or a fragment thereof. Preferably, the PCSK9 comprises the mutation S127R, wherein the amino acids are numbered with reference to SEQ ID NO: 4.

In some embodiments, the nucleotide sequence encoding PCSK9 comprises or consists of the nucleotide sequence of SEQ ID NO: 1, or a fragment thereof.

A fragment and/or variant of PCSK9 may retain PCSK9 activity (e.g. the activity of SEQ ID NO: 4 or 3). For example, a fragment and/or variant of PCSK9 may act as a negative modulator of LDLR and enhance its degradation upon binding. Suitably, a fragment and/or variant of PCSK9 may have the same or similar activity to PCSK9, for example may have at least 40%, at least 50%, at least 60%, at least 70%, at least 80%, at least 90%, at least 95% or 100% of the activity of PCSK9 (e.g. the PCSK9 of SEQ ID NO: 4 or 3). The skilled person will be able to generate fragments and/or variants, for example using conservative substitutions, based on the known structural and functional features of PCSK9.

A “heterologous” polynucleotide (e.g. a heterologous polynucleotide comprising a nucleotide sequence encoding PCSK9) may be a polynucleotide that is not naturally present in the cell, for example has been introduced into the cell by any suitable method, such as transduction or transfection. The heterologous polynucleotide may be, for example, a vector, such as an expression vector. The heterologous polynucleotide may be, for example, a plasmid.

In some embodiments, the nucleotide sequence encoding PCSK9 is operably linked to a promoter. The promoter may be, for example a CMV promoter.

An example promoter sequence is:

(SEQ ID NO: 30)
GGCATTGATTATTGACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTCCGCCCCCTATT
GACGTCAATGACGGTAAATGGCCCGCCTGGCATTATGCCCAGTACATGACCTTACGGGACTTTCCTAC
TTGGCAGTACATCTACGTATTAGTCATCGCTATTACCATGGTGATGCGGTTTTGGCAGTACACCAATG
GGCGTGGATAGCGGTTTGACTCACGGGGATTTCCAAGTCTCCACCCCATTGACGTCAATGGGAGTTTG
TTTTGGCACCAAAATCAACGGGACTTTCCAAAATGTCGTAATAACCCCGCCCCGTTGACGCAAATGGG
CGGTAGGCGTGTACGGTGGGAGGTCTATATAAGCAGAGGT

In some embodiments, the nucleotide sequence encoding PCSK9 is operably linked to a polyadenylation signal. The polyadenylation signal may be, for example, an SV40 polyadenylation signal.

An example polyadenylation signal is:

(SEQ ID NO: 31)
GCAGTGAAAAAAATGCTTTATTTGTGAAATTTGTGATGCTATTGCTTTAT
TTGTAACCATTATAAGCTGCAATAAACAAGTT

In some embodiments, the nucleotide sequence encoding PCSK9 is operably linked to a chimeric intron.

An example chimeric intron sequence is:

(SEQ ID NO: 32)
GTAAGTATCAAGGTTACAAGACAGGTTTAAGGAGGCCAATAGAAACTGGG
CTTGTCGAGACAGAGAAGATTCTTGCGTTTCTGATAGGCACCTATTGGTC
TTACTGACATCCACTTTGCCTTTCTCTCCACAG

### Method of Production

In one aspect, the present invention provides a method of producing enveloped viral particles (e.g. lentiviral particles).

The method of production may comprise: (a) introducing a transfer vector and optionally one or more helper vector into a cell (e.g. a host cell); (b) culturing the cell under conditions suitable to produce enveloped viral particles (e.g. lentiviral particles) according to the present invention. The enveloped viral particles (e.g. lentiviral particles) may then be obtained from the cell.

As used herein, a “transfer vector” may encode the viral (e.g. lentiviral) genome of the present invention. Suitably, the transfer vector used to produce the lentiviral genome within a host cell/packaging cell will have sufficient lentiviral genetic information to allow packaging of an RNA genome, in the presence of packaging components (e.g. gag-pol, rev, env), into a viral particle which is capable of infecting a target cell, but is incapable of independent replication to produce infectious viral particles within the final target cell.

The transfer vector used to produce the viral genome within a host cell/packaging cell may include transcriptional regulatory control sequences operably linked to the lentiviral genome to direct transcription of the genome in a cell. These regulatory sequences may be the natural sequences associated with the transcribed viral sequence (i.e. the 5′ U3 region), or they may be a heterologous promoter, such as another viral promoter (e.g. the CMV promoter). The transfer vector may be a plasmid.

As used herein, a “helper vector” may encode one or more packaging components (e.g. gag-pol, rev, env). The nucleotide sequence encoding the packaging component(s) may be operably linked to a promoter (e.g. a CMV promoter or a RSV promoter) and/or a polyadenylation signal. The term “helper vector” may include “packaging vectors” (e.g. encoding gag-pol or rev) and “envelope vectors” (e.g. encoding an env gene, such as VSV-g). The helper vectors, packaging vectors and/or envelope vectors may be plasmids.

In another aspect, the present invention provides a method of producing enveloped virus-like particles (VLPs) (e.g. lentivirus VLPs).

In another aspect, the invention provides a method of producing enveloped virus-like particles (VLPs) comprising the steps: (a) introducing one or more helper vector into a cell; (b) introducing a vector comprising a nucleotide sequence encoding pro-protein convertase subtilisin/Kexin type 9 (PCSK9) into the cell; and (b) culturing the cell under conditions suitable for the production of the enveloped VLPs.

The transfer vector and/or one or more helper vector may be introduced into the host cell by any suitable technique known in the art, such as transfection, transduction and/or transformation. Suitably, the helper vectors may be transiently transfected or transduced into the host cell or may be stably maintained (e.g. stably integrated into the cell genome) within the host cell. Alternatively, a combination of transient transfection or transduction and stable maintenance may be used to introduce the helper vectors into the host cell.

Suitably, the transfer vector and/or the helper vectors may be plasmids and introduced by transfection. Suitably, a four plasmid system may be used consisting of a transfer plasmid and three helper plasmids. The three helper plasmids may consist of: a first helper plasmid encoding a gag-pol gene; a second helper plasmid encoding a rev gene; and a third helper plasmid encoding an env gene. Alternatively, a three plasmid system may be used which consists of a transfer plasmid, one helper plasmid encoding a gag-pol gene and a rev gene; and one helper plasmid encoding an env gene. Alternatively, a two plasmid system may be used in which all helper functions (e.g. gag-pol, rev and env) are encoded by one helper plasmid.

Any suitable host cell may be used to produce the enveloped viral particles (e.g. lentiviral particles). Any suitable host cell may be used to produce the enveloped VLPs (e.g. lentivirus VLPs). Suitable cells include producer cells and packaging cells, such as those described herein (e.g. HEK 293 or derivatives thereof). Suitable conditions for culturing the cell will be well known to the skilled person. For example, the cells may be incubated in culture medium (e.g. chemically defined medium) for from about 1 day to about 5 days (e.g. about 48 hours, about 54 hours or about 72 hours). For example, the cells may be incubated in culture medium (e.g. chemically defined medium) for from about 2 days to about 3 days.

The enveloped viral particles (e.g. lentiviral particles) may be obtained using any suitable methods known in the art. For example the culture supernatant may be harvested and enveloped viral particles (e.g. lentiviral particles) subsequently purified from the culture supernatant (e.g. by centrifugation, membrane filtration and/or chromatography). The method of production may further comprise any other suitable process steps, for example DNA reduction, concentration, formulation and/or sterilisation.

The enveloped VLPs (e.g. lentivirus VLPs) may be obtained using any suitable methods known in the art. For example the culture supernatant may be harvested and enveloped VLPs (e.g. lentivirus VLPs) subsequently purified from the culture supernatant (e.g. by centrifugation, membrane filtration and/or chromatography). The method of production may further comprise any other suitable process steps, for example DNA reduction, concentration, formulation and/or sterilisation.

### Kits and Systems

In one aspect, the present invention provides a kit or system for producing the enveloped viral particles (e.g. lentiviral particles) of the present invention.

The kit or system may comprise an enveloped viral particle (e.g. lentiviral particle) producer or packaging cell of the invention. The kit or system may further comprise a transfer vector encoding the lentivirus genome of the present invention and optionally one or more helper vector. The kit or system may further comprise other reagents (e.g. transfection reagent, culture medium, etc.). The kit or system may further comprise any other suitable components, and optionally instructions for producing the enveloped viral particles (e.g. lentiviral particles) of the present invention.

In one aspect, the present invention provides a kit or system for producing the enveloped VLPs (e.g. lentivirus VLPs) of the present invention.

The kit or system may comprise an enveloped viral particle packaging cell of the invention. The kit or system may further comprise one or more helper vector. The kit or system may further comprise other reagents (e.g. transfection reagent, culture medium, etc.). The kit or system may further comprise any other suitable components, and optionally instructions for producing the enveloped VLPs (e.g. lentivirus VLPs) of the present invention.

### Cells

The cell may be an isolated cell. Suitably, the cell is a mammalian cell, for example a human cell. The cell may be an isolated human cell.

Suitably, the cell may be a producer cell. The term “producer cell” may refer to a cell that produces viral particles, for example has been transiently transfected, stably transfected and/or transduced with all the elements necessary to produce the viral particles. Suitable producer cells will be well known to the skilled person and may include HEK293, COS-1, COS-7, CV-1, HeLa, CHO and A549 cell lines. In some embodiments, the producer cell is a HEK293 cell, or a derivative thereof (e.g. a HEK293T cell, a HEK293T Lenti-X, a HEK293T-Rex cell, a HEK293FT cell, a HEK293SF-3F6 cell, a HEK293SF-3F9 cell, a HEK293-EBNA1 cell or a SJ293TS cell).

Suitably, the cell may be a packaging cell. The term “packaging cell” may refer to a cell which contains some or all of the elements necessary for packaging a recombinant virus genome. Typically, such packaging cells contain one or more vectors which are capable of expressing viral structural proteins (e.g. gag-pol, rev, env) and/or one or more genes encoding the viral structural proteins have been integrated into the genome of the packaging cell. Cells comprising only some of the elements required for the production of enveloped viral particles are useful as intermediate reagents in the generation of viral particle producer cell lines, through subsequent steps of transient transfection, transduction or stable integration of each additional required element. These intermediate reagents are encompassed by the term “packaging cell”.

Packaging cells lacking a transfer vector may be used for the production of enveloped VLPs as they do not comprise a sequence encoding a viral genome.

### Enveloped Viral Particles

A vector is a tool that allows or facilitates the transfer of an entity from one environment to another. The viral particles of the present invention may be vectors.

The viral vector particles of the invention are enveloped viral particles.

An enveloped viral particle comprises an outer lipid bilayer membrane. Numerous enveloped viruses are known in the art, including lentivirus, retrovirus, herpes simplex virus, vaccinia virus, hepadnavirus, togavirus, flavivirus, arenavirus, coronavirus, orthomyxovirus, paramyxovirus, bunyavirus, bornavirus, rhabdovirus and filovirus.

The enveloped viral particle producer or packaging cell of the invention and the methods of producing enveloped viral particles of the invention may be for production of an enveloped viral particle (preferably a lentiviral particle) as disclosed herein.

### Lentiviral Vector

A lentiviral vector may be in the form of a lentiviral particle. In some embodiments, a lentiviral vector may comprise a lentiviral genome. As used herein, a lentiviral genome may refer to a genome that comprises at least one element derived or derivable from a lentivirus genome.

Lentivirus is a genus of retroviruses, which contain an RNA genome that is converted to DNA in the transduced cell by a reverse transcriptase. Lentiviral vectors can transduce a wide range of cell types and integrate into the host genome in both dividing and post-mitotic cells, resulting in long-term expression of the protein-coding sequence both in vitro and in vivo.

The basic genes required for lentivirus survival and function are the gag, pol, and env genes: gag encodes structural proteins; pol encodes enzymes required for reverse transcription and integration into the host cell genome; and env encodes the viral envelope glycoprotein.

Lentiviruses may also have additional cis-acting elements, such as a rev response element (RRE), which enables the efficient export of RNA transcripts of the integrated provirus from the nucleus to the cytoplasm of an infected target cell; a retroviral psi packaging element, which is involved in regulating the essential process of packaging the retroviral RNA genome into the viral capsid during replication; a primer binding site (PBS), where reverse transcription is initiated; the TAT activation region (TAR); splice donor and acceptor sites; and central and terminal polypurine tracts, which allow initiation of plus-strand synthesis.

In a lentivirus genome, the elements are typically flanked at both ends by regions called long terminal repeats (LTRs). The LTRs are responsible for integration and transcription. LTRs may also serve as enhancer-promoter sequences and can control the expression of the lentiviral genes. The LTRs themselves are identical or near-identical sequences that can typically be divided into three regions: U3, R and U5. LTRs may be naturally occurring or may be modified. For example, U3 and U5 modifications are described in Iwakuma et al. (1999) Virology 261: 120-132.

The lentiviral particle of the present invention may comprise a minimal lentiviral genome. As used herein, a minimal lentiviral genome may mean that the lentiviral genome has been manipulated so as to remove the non-essential elements and to retain the essential elements in order to provide the required functionality to infect, transduce and deliver a nucleotide sequence of interest to a target host cell (see, for example, Kim et al. (1998) Journal of Virology 72: 811-816; Sertkaya et al. (2021) Scientific Reports 11: 1-15).

A lentiviral genome may comprise from 5′ to 3′: a 5′ LTR, one or more lentiviral-derived cis-acting elements, and a 3′ LTR. Suitably, a lentiviral genome may comprise from 5′ to 3′: a 5′ LTR, a RRE, and a 3′ LTR. Suitably, a lentiviral genome may comprise from 5′ to 3′: a 5′ LTR, a retroviral psi packaging element, a RRE, and a 3′ LTR. Suitably, a lentiviral genome may comprise from 5′ to 3′: a 5′ LTR, a retroviral psi packaging element, a RRE, a cPPT, and a 3′ LTR. Suitably, a lentiviral genome may comprise from 5′ to 3′: a 5′ LTR, a PBS, a retroviral psi packaging element, a RRE, a cPPT, and a 3′ LTR.

A lentiviral genome may further comprise a protein-coding sequence and, optionally, one or more regulatory elements (e.g. operably linked to the protein-coding sequence). Suitably, a lentiviral genome may comprise from 5′ to 3′: a 5′ LTR, a RRE, a protein-coding sequence, and a 3′ LTR. Suitably, a lentiviral genome may comprise from 5′ to 3′: a 5′ LTR, a retroviral psi packaging element, a RRE, a protein-coding sequence, and a 3′ LTR. Suitably, a lentiviral genome may comprise from 5′ to 3′: a 5′ LTR, a retroviral psi packaging element, a RRE, a cPPT, a protein-coding sequence, and a 3′ LTR. Suitably, lentiviral genome may comprise from 5′ to 3′: a 5′ LTR, a PBS, a retroviral psi packaging element, a RRE, a cPPT, a protein-coding sequence, and a 3′ LTR.

The lentiviral particle of the present invention may be replication-defective. Typically, at least part of one or more protein coding regions essential for replication may be removed from the lentiviral genome. This makes the lentivirus “replication-defective” or “replication-incompetent”. Suitably, one or more of gag, pol, rev, and env genes are deleted (at least partially) in a replication-defective lentivirus. Suitably, each of the gag, pol, rev, and env genes are deleted (at least partially) in a replication-defective lentivirus. Optionally, the lentivirus lacks a functional gag-pol and/or env gene and/or other genes essential for replication.

The lentiviral particle of the present invention may be derived from any lentivirus. As used herein “lentivirus-derived” or “lentivirus-based” may mean that the lentiviral genome comprises one or more elements from said lentivirus. For example, the coding regions of viral proteins may be deleted, but one or more cis-acting element may be retained from said lentivirus.

The lentiviral particle may be derived from a primate lentivirus. Examples of “primate” lentiviruses include, but are not limited to, human immunodeficiency virus (HIV) and simian immunodeficiency virus (SIV). The lentiviral particle may be derived from a non-primate lentivirus (i.e. derived from a lentivirus which does not primarily infect primates, especially humans). Examples of “non-primate” lentiviruses include, but are not limited to, the prototype “slow virus” visna/maedi virus (VMV), caprine arthritis-encephalitis virus (CAEV), equine infectious anaemia virus (EIAV), feline immunodeficiency virus (FIV), and bovine immunodeficiency virus (BIV).

Suitably, the lentiviral particle of the present invention is a HIV-derived lentiviral particle. As used herein “HIV-derived” or “HIV-based” may mean that the lentiviral genome comprises one or more element from HIV. For example, the coding regions of HIV viral proteins may be deleted, and one or more HIV cis-acting element may retained in the lentiviral genome (see, for example, Johnson (2021) Molecular Therapy-Methods & Clinical Development 21: 451-465). A HIV-derived lentiviral genome may comprise from 5′ to 3′: a 5′ LTR, one or more HIV-derived cis-acting elements (e.g. RRE and/or cPPT), and a 3′ LTR.

Suitably, the lentiviral particle of the present invention is a HIV-1-derived lentiviral particle. The prototype lentiviral vector system is based on HIV-1 (see, for example, Merten et al. (2016) Molecular Therapy-Methods & Clinical Development 3: 16017). It has been shown that sequences that extend into the gag open reading frame may be important for packaging of HIV-1. Therefore, HIV-1 vectors often contain the relevant portion of gag in which the translational initiation codon has been mutated. In addition, HIV-1 vectors often also contain a portion of the env gene that includes the RRE. Rev binds to RRE, which permits the transport of full-length or singly spliced mRNAs from the nucleus to the cytoplasm. In the absence of rev and/or a RRE, full-length HIV-1 RNAs may accumulate in the nucleus. Alternatively, a constitutive transport element from certain simple retroviruses such as Mason-Pfizer monkey virus can be used to relieve the requirement for rev and a RRE. A HIV-1-derived lentiviral genome may comprise from 5′ to 3′: a 5′ LTR, one or more HIV-1-derived cis-acting elements (e.g. a PBS, a retroviral psi packaging element, a RRE and/or a cPPT), and a 3′ LTR.

The lentiviral particle of the present invention may be a self-inactivating lentiviral particle. As used herein, “self-inactivating” or “SIN” lentiviral particle may comprise lentiviral genomes in which the lentiviral enhancer and promoter sequences have been deleted (see, for example, Zufferey (1998) Journal of Virology 72: 9873-9880; Miyoshi et al. (1998) Journal of Virology 72: 8150-8157). SIN lentiviral particles can be generated and transduce non-dividing cells in vivo with an efficacy similar to that of wild-type vectors. The transcriptional inactivation of the long terminal repeat (LTR) in the SIN provirus can prevent mobilisation by replication-competent virus. This can also enable the regulated expression of genes from internal promoters by eliminating any cis-acting effects of the LTR.

The lentiviral vector particle of the present invention may be integration competent. As used herein, an “integration competent” lentiviral vector is capable of integrating into the genome of a host cell. In contrast to integration competent lentiviral vectors, integration defective lentiviral vectors (IDLVs) can be produced, for example by packaging the lentiviral vector with catalytically inactive integrase (such as an HIV integrase bearing the D64V mutation in the catalytic site) or by modifying or deleting essential att sequences from the lentiviral genome LTR, or by a combination of the above (see, for example. Wanisch et al. (2009) Molecular Therapy 17: 1316-1332).

The lentiviral particle of the present invention may be replication-defective and integrating. The lentiviral particle of the present invention may be replication-defective, integrating, and self-inactivating. The lentiviral particle of the present invention may be replication-defective, integrating, self-inactivating, and HIV-derived.

A “lentiviral particle” may refer to an enveloped lentiviral genome. Lentiviral particles may be generated by co-transfection of a plasmid containing a lentiviral genome (e.g. a “transfer vector”) with helper plasmids (e.g. “packaging vectors” encoding gag-pol and/or rev, and “envelope vectors” encoding env) into host cells and harvesting of the lentivirus-containing supernatant afterwards.

The lentiviral particle of the present invention may be pseudotyped. Pseudotyping lentiviral particle with naturally occurring or engineered lentiviral envelopes can allow targeted transduction of specific cell types (see, for example, Joglekar et al. (2017) Human Gene Therapy Methods 28: 291-301).

Preferably, the lentiviral particle of the present invention is VSV-G pseudotyped. Vesicular stomatitis virus G protein (VSV-G) is a commonly used envelope protein for pseudotyping. VSV-G is a trimeric protein that binds phosphatidylserine and low-density lipoprotein receptors on a cell surface to endocytose into the cell.

The lentiviral particle of the present invention may be replication-defective, integrating, and VSV-G pseudotyped. The lentiviral particle of the present invention may be replication-defective, integrating, self-inactivating, and VSV-G pseudotyped. The lentiviral particle of the present invention may be replication-defective, integrating self-inactivating, HIV-derived, and VSV-G pseudotyped.

In some embodiments, the lentiviral particle of the present invention: (i) comprises one or more miRNA target sequence; (ii) is a CD47^{high} lentiviral particle; and/or (iii) is a MHC-I^{free} lentiviral particle. In preferred embodiments, the lentiviral particle of the present invention comprises one or more miRNA target sequence and is a CD47^{high}/MHC-I^{free} lentiviral particle. Each of these features may reduce immune responses following administration.

In some embodiments, the enveloped viral particle producer or packaging cell of the present invention is a CD47^{high} producer or packaging cell. In some embodiments, the enveloped viral particle producer or packaging cell of the present invention is a MHC-I^{low} producer or packaging cell. In some embodiments, the enveloped viral particle producer or packaging cell of the present invention is a MHC-I^{free} producer or packaging cell.

The enveloped viral particle producer or packaging cell of the present invention may be a CD47^{high}/MHC-I^{free} producer or packaging cell or a CD47^{highh}/MHC-I^{low} producer or packaging cell. In preferred embodiments, the enveloped viral particle producer or packaging cell of the present invention is a CD47^{high}/MHC-I^{free} producer or packaging cell.

miRNA Target Sequence

The lentiviral particle of the present invention may comprise one or more miRNA target sequence. The one or more miRNA target sequence may be operably linked to the protein-coding sequence. The term “operably linked” may mean that the components described are in a relationship permitting them to function in their intended manner.

MicroRNA (miRNA) genes are scattered across all human chromosomes, except for the Y chromosome. Similar to protein-coding genes, miRNAs are usually transcribed from polymerase-II promoters, generating a so-called primary miRNA transcript (pri-miRNA). From the pri-miRNA, a stem loop of about 60 nucleotides in length, called miRNA precursor (pre-miRNA), is excised leaving a 5′ phosphate and a 2 bp long, 3′ overhang. The pre-miRNA is then actively transported from the nucleus to the cytoplasm. Then, Dicer performs a double strand cut at the other end of the stem loop, generating a 19-24 bp duplex, which is composed of the mature miRNA and the opposite strand of the duplex, called miRNA*. One strand of the duplex is selectively loaded into the RNA-induced silencing complex (RISC), and accumulates as the mature microRNA. This strand is usually the one whose 5′ end is less tightly paired to its complement. However, there are some miRNAs that support accumulation of both duplex strands to similar extent.

Once loaded into RISC, the guide strand of the mature microRNA interacts with mRNA target sequences preferentially found in the 3′ untranslated region (3′UTR) of protein-coding genes. If the whole guide strand sequence is perfectly complementary to the mRNA target, the mRNA is endonucleolytically cleaved. If only the seed sequence (i.e. nucleotides 2-8 counted from the 5′ end of the miRNA) is perfectly complementary to the target mRNA, RNAi may act through alternative mechanisms leading to translational repression.

Expression of the protein from the protein-coding sequence (i.e. “transgene expression”) may be regulated by one or more endogenous miRNAs using one or more corresponding miRNA target sequence. Using this method, one or more miRNAs endogenously expressed in a cell prevent or reduce transgene expression in that cell by interacting with its corresponding miRNA target sequence positioned in the lentiviral genome.

Suitable miRNA target sequences which suppress transgene expression in specific cells will be known to the skilled person. Determining a miRNA with the desired expression profile may be achieved using techniques known to those skilled in the art. For example, a mammalian microRNA expression atlas is described in Landgraf et al. (2007) Cell 129: 1401-1414 and the distribution of miRNA expression across human tissues is described in Ludwig et al. (2016) Nucleic Acids Research 44: 3865-3877. Once a miRNA has been identified, the corresponding target sequence can readily be determined using, for example, a microRNA database, such as miRBase (Griffiths-Jones et al. (2007) Nucleic Acids Research 36(suppl_1): D154-D158).

A miRNA target sequence may be fully or partially complementary to the corresponding miRNA. The term “fully complementary”, as used herein, may mean that the target sequence has a nucleic acid sequence which is 100% complementary to the sequence of the miRNA which recognises it. The term “partially complementary”, as used herein, may mean that the target sequence is only in part complementary to the sequence of the miRNA which recognises it, whereby the partially complementary sequence is still recognised by the miRNA. In other words, a partially complementary target sequence in the context of the present invention is effective in recognising the corresponding miRNA and effecting prevention or reduction of transgene expression in cells expressing that miRNA. Suitably, a partially complementary miRNA target sequence may be fully complementary to the miRNA seed sequence.

Including more than one copy of a miRNA target sequence in a lentiviral vector may increase the effectiveness of the system. Also, different miRNA target sequences can be included. For example, the protein-coding sequence may be operably linked to more than one miRNA target sequence, which may or may not be different. The miRNA target sequences may be in tandem, but other arrangements are envisaged. The lentiviral vector may, for example, comprise 1, 2, 3, 4, 5, 6, 7 or 8 copies of the same or different miRNA target sequences. Suitably, the lentiviral vector comprises 4 miRNA target sequences of each miRNA target sequence.

Copies of miRNA target sequences may be separated by a spacer sequence. A spacer sequence may comprise, for example, at least one, at least two, at least three, at least four or at least five nucleotide bases.

Suitably, the lentiviral vector comprises one or more miRNA target sequence, two or more miRNA target sequences, three or more miRNA target sequences, or four or more miRNA target sequences. Suitably, the protein-coding sequence is operably linked to one or more miRNA target sequence, two or more miRNA target sequences, three or more miRNA target sequences, or four or more miRNA target sequences. In some embodiments, the protein-coding sequence is operably linked to four miRNA target sequences.

The miRNA target sequence may be a human miRNA target sequence. Suitably, the miRNA target sequence is a −5p or −3p miRNA target sequence.

The one or more miRNA target sequence may suppress transgene expression in one or more cells other than liver cells (e.g. hepatocytes).

The one or more miRNA target sequence may suppress transgene expression in hematopoietic-lineage cells. Hematopoietic stem cells give rise to different types of blood cells, in lines called myeloid and lymphoid. As used herein, “hematopoietic-lineage cells” may include myeloid cells and lymphoid cells. Myeloid cells may include monocytes, macrophages, neutrophils, basophils and eosinophils. Lymphoid cells may include T cells, B cells, natural killer cells and innate lymphoid cells.

The one or more miRNA target sequence may suppress transgene expression in antigen-presenting cells. As used herein, an “antigen presenting cell” (APC) may refer to a cell that displays antigen bound by major histocompatibility complex (MHC) proteins on its surface.

APCs may be hematopoietic-lineage cells. The antigen-presenting cells may be professional antigen-presenting cells. Professional APCs specialize in presenting antigens to T cells and may include macrophages, B cells and dendritic cells. Suitably, the APCs are splenic and/or hepatic APCs.

The one or more miRNA target sequence may suppress transgene expression in hematopoietic-lineage antigen-presenting cells.

By preventing transgene expression in antigen-presenting cells, while permitting high levels of expression in other cells, miRNA regulation may enable strong and stable gene transfer in the absence of an immune response.

As used herein, the term “suppress expression” may refer to a reduction of expression in the relevant cell type(s) of a transgene to which the one or more miRNA target sequence is operably linked as compared to transgene expression in the absence of the one or more miRNA target sequence, but under otherwise substantially identical conditions. In some embodiments, transgene expression is suppressed by at least 50%. In some embodiments, transgene expression is suppressed by at least 60%, at least 70%, at least 80%, at least 90%, or at least 95%. In some embodiments, transgene expression is substantially prevented, for example is not detectable.

The miRNA-mediated approach for restricting gene expression has several advantages over other strategies of regulating transgenes. Although using tissue-specific promoters can successfully limit expression to target cells, leaky expression in a fraction of non-target cells is observed. This occurs because the reconstituted promoter, modified for inclusion into a vector system, often loses some of its cell specificity and also because vector integration near active promoters and enhancers can activate the tissue-specific promoter and drive transgene expression. In contrast, because miRNA-mediated silencing occurs at the post-transcriptional level, promoter and enhancer trapping is irrelevant. As such, miRNA-regulation can be used to effectively de-target transgene expression from a particular cell type, while still allowing for broad tissue expression. miRNA regulation may also be used as in combination with tissue-specific promoter/enhancers. By including the miRNA target sequence in expression cassettes already under the control of a tissue-specific promoter, an additional layer of regulation is added which may eliminate off-target expression.

Exemplary miRNA target sequences which suppress transgene expression in hematopoietic-lineage cells and/or antigen-presenting cells, include, but are not limited to, miR-142, miR-181, miR-223 and miR-155 target sequences. Other miRNA target sequences which suppress transgene expression in hematopoietic-lineage cells and/or antigen-presenting cells are known in the art (see, for example, Ghafouri-Fard et al. (2021) Non-coding RNA research 6: 8-14). miRNAs which are expressed in hematopoietic-lineage cells and/or antigen-presenting cells interact with the corresponding miRNA target sequence and reduce the expression of the target gene.

Further miRNA target sequences that suppress transgene expression in hematopoietic-lineage cells and/or antigen-presenting cells can be identified by any suitable method, for example miRNA expression analysis as described in Monticelli et al. (2005) Genome Biology 6: 1-15.

### CD47^{high} Lentiviral Particles

The lentiviral particle of the present invention may be a CD47^{high} lentiviral particle. As used herein, a “CD47^{high} lentiviral particle” may refer to a lentiviral particle with increased levels of CD47 (or a fragment thereof) on its surface. A CD47^{high} lentiviral particle may have reduced uptake by professional phagocytes. In some embodiments the surface of a CD47^{high} lentiviral particle comprises a higher level of CD47 protein than a control lentiviral particle produced in HEK293T cells (AT CC® CRL-1 1268™)

CD47 (Cluster of Differentiation 47) also known as integrin associated protein (IAP) is a transmembrane protein that in humans is encoded by the CD47 gene. Phagocytosis is physiologically inhibited by CD47, which is a ubiquitously expressed ligand of signal regulatory protein a (SIRP-α) receptor, that is expressed by professional phagocytes. CD47 may be incorporated into lentiviral particles when they bud from producer cells.

The lentiviral particle of the present invention may comprise one or more CD47 polypeptides (or a fragment thereof) on its surface. The amount of CD47 (or a fragment thereof) on the surface may be enough to reduce uptake by professional phagocytes. Any suitable assay to quantify the amount of CD47 polypeptides (or fragments thereof) present on the surface of the lentiviral particle may be used.

In some embodiments, the density of CD47 polypeptides (or fragments thereof) may be determined by immunostaining for CD47 and total internal reflection fluorescence microscopy, for example as described in US20100316570A1. The CD47 polypeptides (or fragments thereof) may be present in a density of at least about 20 molecules/μm², at least about 25 molecules/μm², at least about 30 molecules/μm², at least about 35 molecules/μm², at least about 40 molecules/μm², at least about 45 molecules/μm², at least about 50 molecules/μm², at least about 60 molecules/μm², at least about 70 molecules/μm², at least about 80 molecules/μm², at least about 90 molecules/μm², at least about 100 molecules/μm², at least about 150 molecules/μm², at least about 200 molecules/μm², at least about 250 molecules/μm², at least about 300 molecules/μm², at least about 350 molecules/μm², at least about 400 molecules/μm², at least about 450 molecules/μm², at least about 500 molecules/μm², at least about 600 molecules/μm², at least about 700 molecules/μm², at least about 800 molecules/μm², at least about 900 molecules/μm² or at least about 1000 molecules/μm². The CD47 polypeptides (or fragments thereof) may be present in a density of about 1000 molecules/μm² or less, about 500 molecules/μm² or less or about 250 molecules/μm² or less. The CD47 polypeptides (or fragments thereof) may be present in a density of from about 20 molecules/μm² to about 1000 molecules/μm², from about 20 molecules/μm² to about 500 molecules/μm² or from about 20 molecules/μm² to about 250 molecules/μm².

In some embodiments, the amount of CD47 polypeptides (or fragments thereof) may be determined by immunostaining for CD47 and electron microscopy, as described in Milani et al. (2019) Science Translational Medicine 11: eaav7325. The CD47 polypeptides (or fragments thereof) may be detected in an amount of at least about 10 gold particles/lentiviral particle, at least about 15 gold particles/lentiviral particle or at least about 20 gold particles/lentiviral particle. The CD47 polypeptides (or fragments thereof) may be detected in an amount of about 100 gold particles/lentiviral particle or less, about 80 gold particles/lentiviral particle or less or about 60 gold particles/lentiviral particle or less. The CD47 polypeptides (or fragments thereof) may be detected in an amount of from about 10 to about 100 gold particles/lentiviral particle, from about 15 to about 80 gold particles/lentiviral particle or from about 20 to about 60 gold particles/lentiviral particle.

The lentiviral particle of the present invention may be obtained from a CD47^{high} producer cell. As used herein, a “CD47^{high} producer cell” may refer to a producer cell with increased levels of CD47 (or a fragment thereof) on its surface.

A CD47^{high} producer cell may be genetically engineered to increase expression of CD47 (or a fragment thereof) on the cell surface. For example, the producer cell may comprise a vector encoding CD47 (or a fragment thereof) or may be edited to introduce a nucleotide sequence encoding CD47 (or a fragment thereof) into its genome. Suitably, the producer cell is transduced with a viral vector encoding a CD47 polypeptide (or a fragment thereof).

A CD47^{high} producer cell may have a higher concentration of CD47 (or a fragment thereof) on its surface than an unmodified producer cell. Suitably, the producer cell has at least about 2-fold, at least about 3-fold, at least about 4-fold, at least about 5-fold, at least about 10-fold, at least about 20-fold or at least about 30-fold more CD47 on its cell surface than an unmodified producer cell. Suitably, the producer cell has from about 5-fold to about 30-fold more CD47 (or a fragment thereof) on its cell surface than an unmodified producer cell.

Suitably, the lentiviral particle of the present invention has a higher concentration of CD47 (or a fragment thereof) on its surface than a lentiviral particle obtained from an unmodified producer cell. Suitably, the lentiviral particle has at least about 2-fold, at least about 3-fold, at least about 4-fold, at least about 5-fold, at least about 10-fold, at least about 20-fold, at least about 30-fold, at least about 40-fold or at least about 50-fold more CD47 (or a fragment thereof) on its surface than a lentiviral particle obtained from an unmodified producer cell. Suitably, the lentiviral particle has from about 5-fold to about 30-fold more CD47 (or a fragment thereof) on its surface than a lentiviral particle obtained from an unmodified producer cell.

CD47 is a member of the immunoglobulin (Ig) superfamily of membrane proteins, with a single IgV-like domain at its N-terminus, a highly hydrophobic stretch with five membrane-spanning segments and an alternatively spliced cytoplasmic C-terminus ranging in length from 3 to 36 amino acids. Mouse, rat, bovine and human CD47 molecules have been cloned and show about 70% overall amino acid identity (see, for example, Brown et al. (2001) Trends Cell Biology 11: 130-135).

The CD47 polypeptide (or a fragment thereof) may be a human CD47 polypeptide (or a fragment thereof). A CD47 polypeptide may have an amino acid sequence of UniProtKB Q08722.

Exemplary CD47 polypeptides are provided by SEQ ID NOs: 6-9. Suitably, a CD47 polypeptide comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to any of SEQ ID NOs: 6-9. Suitably, a CD47 polypeptide comprises or consists of the amino acid sequence of any of SEQ ID NOs: 6-9.

### Example CD47 Amino Acid Sequences:

(SEQ ID NO: 6)
MWPLVAALLLGSACCGSAQLLENKTKSVEFTFCNDTVVIPCFVTNMEAQNTTEVYVKWKFKGRDIYTE
DGALNKSTVPTDESSAKIEVSQLLKGDASLKMDKSDAVSHTGNYTCEVTELTREGETIIELKYRVVSW
ESPNENILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPMHGPLLI
SGLSILALAQLLGLVYMKEVASNQKTIQPPRNN
(SEQ ID NO: 7)
MWPLVAALLLGSACCGSAQLLENKTKSVEFTFCNDTVVIPCFVTNMEAQNTTEVYVKWKEKGRDIYTF
DGALNKSTVPTDESSAKIEVSQLLKGDASLKMDKSDAVSHTGNYTCEVTELTREGETIIELKYRVVSW
FSPNENILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPMHGPLLI
SGLSILALAQLLGLVYMKFVASNQKTIQPPRKAVEEPLNAFKESKGMMNDE
(SEQ ID NO: 8)
MWPLVAALLLGSACCGSAQLLENKTKSVEFTFCNDTVVIPCFVTNMEAQNTTEVYVKWKEKGRDIYTF
DGALNKSTVPTDESSAKIEVSQLLKGDASLKMDKSDAVSHTGNYTCEVTELTREGETIIELKYRVVSW
FSPNENILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPMHGPLLI
SGLSILALAQLLGLVYMKFV
(SEQ ID NO: 9)
MWPLVAALLLGSACCGSAQLLENKTKSVEFTFCNDTVVIPCFVTNMEAQNTTEVYVKWKFKGRDIYTF
DGALNKSTVPTDESSAKIEVSQLLKGDASLKMDKSDAVSHTGNYTCEVTELTREGETIIELKYRVVSW
FSPNENILIVIFPIFAILLFWGQFGIKTLKYRSGGMDEKTIALLVAGLVITVIVIVGAILFVPGEYSL
KNATGLGLIVTSTGILILLHYYVESTAIGLTSFVIAILVIQVIAYILAVVGLSLCIAACIPMHGPLLI
SGLSILALAQLLGLVYMKFVASNQKTIQPPRKAVEEPLN

Exemplary CD47 polypeptides excluding the signal peptide are provided by SEQ ID NOs: 10-13. Suitably, a CD47 polypeptide comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to any of SEQ ID NOs: 10-13. Suitably, a CD47 polypeptide comprises or consists of the amino acid sequence of any of SEQ ID NOs: 10-13.

### Example CD47 Amino Acid Sequences Excluding Signal Peptide:

(SEQ ID NO: 10)
QLLENKTKSVEFTFCNDTVVIPCFVTNMEAQNTTEVYVKWKFKGRDIYTEDGALNKSTVPTDESSAKI
EVSQLLKGDASLKMDKSDAVSHTGNYTCEVTELTREGETIIELKYRVVSWESPNENILIVIFPIFAIL
LFWGQFGIKTLKYRSGGMDEKTIALLVAGLVITVIVIVGAILFVPGEYSLKNATGLGLIVTSTGILIL
LHYYVESTAIGLTSFVIAILVIQVIAYILAVVGLSLCIAACIPMHGPLLISGLSILALAQLLGLVYMK
FVASNQKTIQPPRNN
(SEQ ID NO: 11)
QLLENKTKSVEFTFCNDTVVIPCFVTNMEAQNTTEVYVKWKFKGRDIYTEDGALNKSTVPTDESSAKI
EVSQLLKGDASLKMDKSDAVSHTGNYTCEVTELTREGETIIELKYRVVSWESPNENILIVIFPIFAIL
LFWGQFGIKTLKYRSGGMDEKTIALLVAGLVITVIVIVGAILFVPGEYSLKNATGLGLIVTSTGILIL
LHYYVFSTAIGLTSFVIAILVIQVIAYILAVVGLSLCIAACIPMHGPLLISGLSILALAQLLGLVYMK
FVASNQKTIQPPRKAVEEPLNAFKESKGMMNDE
(SEQ ID NO: 12)
QLLFNKTKSVEFTFCNDTVVIPCFVTNMEAQNTTEVYVKWKFKGRDIYTEDGALNKSTVPTDESSAKI
EVSQLLKGDASLKMDKSDAVSHTGNYTCEVTELTREGETIIELKYRVVSWESPNENILIVIFPIFAIL
LFWGQFGIKTLKYRSGGMDEKTIALLVAGLVITVIVIVGAILFVPGEYSLKNATGLGLIVTSTGILIL
LHYYVFSTAIGLTSFVIAILVIQVIAYILAVVGLSLCIAACIPMHGPLLISGLSILALAQLLGLVYMK
FV
(SEQ ID NO: 13)
QLLENKTKSVEFTFCNDTVVIPCFVTNMEAQNTTEVYVKWKFKGRDIYTEDGALNKSTVPTDESSAKI
EVSQLLKGDASLKMDKSDAVSHTGNYTCEVTELTREGETIIELKYRVVSWESPNENILIVIFPIFAIL
LFWGQFGIKTLKYRSGGMDEKTIALLVAGLVITVIVIVGAILFVPGEYSLKNATGLGLIVTSTGILIL
LHYYVFSTAIGLTSFVIAILVIQVIAYILAVVGLSLCIAACIPMHGPLLISGLSILALAQLLGLVYMK
FVASNQKTIQPPRKAVEEPLN

The skilled person would be able to generate variants and/or fragments, for example based on conservative substitutions and/or the known structural and functional features of CD47. These are described, for instance in Fenalti et al. (2021) Nature Communications 12: 1-14. Suitably, a fragment of CD47 and/or CD47 variant retains the ability to inhibit phagocytosis. Suitably, a CD47 fragment and/or CD47 variant may comprise the extracellular domain of CD47. The extracellular domain of human CD47 may interact with SIRP-α and inhibit phagocytosis. Optionally, a CD47 fragment and/or CD47 variant comprises the transmembrane domain of CD47. The domains may be linked by inter-domain linker(s). The fragment and/or variant may have at least 40%, at least 50%, at least 60%, at least 70%, at least 80%, at least 90%, at least 95% or 100% of the activity of a full-length CD47 polypeptide.

Suitably, a variant of SEQ ID NO: 6 may comprise one or more variation selected from V51, C14W, C15R, F22L, S27F, F30L, F32Y, T36S, V38L, V381, F42V, T44A, N50S, T51A, T52S, T52A, V561, R63K, A71T, S75Y, T76A, P78L, P78S, P78A, S82R, S82N, S83T, K85N, K85E, V88A, V88L, V881, Q90R, L91F, K93N, M1001, M100V, D101G, K102R, K102T, S107L, 1126F, 1127V, K130Q, R132H, S138F, V1461, 1150V, 1153V, S169A, G170R, G170S, G171S, D173Y, 1177V, A178G, V1811, V185A, 1186V, V188A, 1189T, 1191V, V1981, A207S, T2151, 1219M, Y226C, A231S, T235A, S236F, A240V, A240T, 1241V, V2431, 1244T, V246L, Y249F, A252S, A252T, V254A, S257T, 1264M, 1264L, M2661, M266T, M266V, V2871, V292A, N295S, N295D, Q296L, P302S, N304S and N304D. These are considered to be tolerated, benign, and/or likely benign variations as predicted by SIFT, PolyPhen, CADD, REVEL and MetaLR.

Suitably, a variant of SEQ ID NO: 7 may comprise one or more variation selected from P3L, A6P, F22L, S27F, F30L, F32Y, T36S, V38L, V381, F42V, N50S, T51A, T52S, T52A, V561, R63K, A71T, S75Y, T76A, P78L, P78S, P78A, S82R, S82N, S83T, K85N, K85E, V88A, V88L, V881, Q90R, L91F, K93N, M100I, M100V, D101G, K102R, K102T, S107L, 1126F, 1127V, K130Q, R132H, S138F, V1461, 1150V, 1153V, S169A, G170R, G170S, G171S, 1177V, A178G, V1811, 1186V, V188A, 1189T, 1191V, V1981, A207S, T2151, 1219M, Y226C, A231S, T235A, A240V, A240T, 1241V, V2431, 1244T, V246L, Y249F, A252S, A252T, V254A, 1264M, 1264L, M2661, M266T, M266V, V2871, V292A, N295S, N295D and Q296L. These are considered to be tolerated, benign, and/or likely benign variations as predicted by SIFT, PolyPhen, CADD, REVEL, and MetaLR.

An exemplary CD47 fragment is provided by SEQ ID NO: 14. Suitably, a CD47 fragment comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 14. Suitably, a CD47 fragment comprises or consists of the amino acid sequence of SEQ ID NO: 14.

### Example CD47 Fragment:

(SEQ ID NO: 14)
MWPLVAALLLGSACCGSAQLLENKTKSVEFTFCNDTVVIPCFVTNMEAQN
TTEVYVKWKEKGRDIYTFDGALNKSTVPTDESSAKIEVSQLLKGDASLKM
DKSDAVSHTGNYTCEVTELTREGETIIELKYRVVSWESPN

An exemplary CD47 fragment excluding the signal peptide is provided by SEQ ID NO: 15. Suitably, a CD47 fragment comprises an amino acid sequence that has at least about 70%, at least about 75%, at least about 80%, at least about 85%, at least about 90%, at least about 95%, at least about 96%, at least about 97%, at least about 98%, at least about 99% or about 100% identity to SEQ ID NO: 15. Suitably, a CD47 fragment comprises or consists of the amino acid sequence of SEQ ID NO: 15.

### Example CD47 Fragment Excluding Signal Peptide:

(SEQ ID NO: 15)
QLLENKTKSVEFTFCNDTVVIPCFVTNMEAQNTTEVYVKWKFKGRDIYTE
DGALNKSTVPTDESSAKIEVSQLLKGDASLKMDKSDAVSHTGNYTCEVTE
LTREGETIIELKYRVVSWESPN

MHC-I^{low} or MHC-I^{free} Lentiviral Particles

The lentiviral particle of the present invention may be a MHC-I^{low} lentiviral particle or a MHC-I^{free} lentiviral particle. In preferred embodiments, the lentiviral particle of the present invention is a MHC-I^{free} lentiviral particle.

As used herein, a “MHC-I^{low} lentiviral particle” may refer to a lentiviral particle with reduced levels of one or more MHC-I molecules on its surface (i.e. reduced levels of surface-exposed MHC-I molecules). The number of surface-exposed MHC-I molecules may be reduced such that the immune response to the MHC-I is decreased to a therapeutically relevant degree.

As used herein, a “MHC-I^{free} lentiviral particle” may refer to a lentiviral particle which is substantially devoid of (or free of) one or more MHC-I molecules on its surface (i.e. substantially devoid of (or free of) surface-exposed MHC-I molecules). Specifically, the surface of the lentiviral particle may not comprise MHC-I.

The major histocompatibility complex class I (MHC-I) is a heterodimeric membrane protein that is displayed on the outer leaflet of the cell membrane (see, for example, Penn et al. (2005) Major histocompatibility complex (MHC). eLS). MHC-I functions to bind and display peptide fragments of proteins to the extracellular environment where they may be recognised by CD8+ cytotoxic T cells. Peptide fragments generated from normal cellular proteins will not activate cytotoxic T cells due to central and peripheral tolerance mechanisms. However, foreign peptides (e.g. those originating from viral proteins) will cause activation of an immune response to destroy the cell. An allogeneic MHC-1 protein itself may be recognised by the immune system. For example, antibodies may bind MHC-1 epitopes directly. As a result, lentiviral particles that comprise MHC-1 molecules originating from an allogeneic source may be targeted and neutralised by the immune system.

The term “MHC-1 molecules” may refer to human MHC-1 molecules. Human MHC-1 is also referred to as human leukocyte antigen class I (HLA-1) and is expressed on almost all nucleated cells. HLA-I consists of two polypeptide chains, an HLA-I heavy chain (a chain) and P2 microglobulin (P2M or p chain). The HLA-I α chain and P2M are linked non-covalently. The HLA-I α chain is polymorphic. Six HLA-I α chains have been identified to date, including three classical, highly polymorphic a chains (HLA-A, HLA-B and HLA-C) and three non-classical, less polymorphic (HLA-E, HLA-F and HLA-G) a chains. The MHC-1 molecules may comprise or consist of HLA-A, HLA-B and HLA-C molecules, which comprise an invariant P2M sequence.

The term “MHC-1 molecules” may also include variant MHC-1 sequences, such as polymorphisms of HLA-I α chain sequences and/or P2M sequences. For example, variant MHC-1 sequences may include HLA-I α chain sequences and/or P2M sequences with single nucleotide polymorphisms (SNPs) or multiple SNPs.

Any suitable assay to quantify the amount of MHC-1 molecules present on the surface of the lentiviral particle may be used.

In some embodiments, the amount of MHC-1 molecules may be determined by immunostaining for MHC-1 and electron microscopy, for example as described in Milani et al. (2017) EMBO Molecular Medicine 9: 1558-1573. The MHC-1 molecules may be detected in an amount of less than about 10 gold particles/lentiviral particle, less than about 9 gold particles/lentiviral particle, less than about 8 gold particles/lentiviral particle, less than about 7 gold particles/lentiviral particle, less than about 6 gold particles/lentiviral particle, less than about 5 gold particles/lentiviral particle, less than about 4 gold particles/lentiviral particle, less than about 3 gold particles/lentiviral particle, less than about 2 gold particles/lentiviral particle, less than about 1 gold particle/lentiviral particle or about 0 gold particles/lentiviral particle. The MHC-1 molecules may be undetectable (e.g. the amount of gold particles detected may not be significantly higher than background levels).

The lentiviral particle of the present invention may be obtained from a MHC-I^{low} producer cell or a MHC-I^{free} producer cell. In preferred embodiments, the lentiviral particle of the present invention is obtained from a MHC-I^{free} producer cell. As used herein, a “MHC-II^{low} producer cell” may refer to a producer cell with reduced levels of one or more MHC-1 molecule on its surface. As used herein, a “MHC-I^{free} producer cell” may refer to a producer cell which is substantially devoid of or free of one or more MHC-1 molecule on its surface. Specifically, the surface of the lentiviral particle may, for example, not comprise MHC-I.

A MHC-I^{low} or MHC-I^{free} producer cell may be genetically engineered to decrease expression of MHC-1 on the cell surface. For example, the cell may comprise a genetically engineered disruption of a gene encoding P2-microglobulin and/or a genetically engineered disruption of a gene encoding an MHC-1 a chain.

Methods for genetic engineering to decrease protein expression are known in the art. For example, this may be achieved by targeted gene knockout. To decrease protein expression, the gene encoding the protein itself or its regulatory sequence (e.g. its promoter) may be knocked out. Knockout may be achieved by deletion of a section of the coding nucleic acid sequence, which may delete a section of the protein essential for expression or stability, or alter the reading frame of the coding sequence or by base-editing. Suitable methods for targeted gene knockout include use of zinc finger nucleases (ZFNs), transcription activator-like effector nucleases (TALENs) and CRISPR/Cas-based RNA-guided nucleases (see e.g. Gaj et al. (2013) Trends Biotechnol 31: 397-405). For example, the CRISPR/Cas9 RNA-guided nuclease may be used to catalyse a double strand break at a specific locus in the genome if provided with appropriate RNA guides designed to bind that locus. Cas9 and the guide RNA may be delivered to a target cell by transfection of vectors encoding the protein and RNA. Cells attempt to repair any double strand breaks in their DNA using the non-homologous end joining (NHEJ) pathway. This is an error-prone mechanism which inserts random nucleotides and often disrupts the reading frame of the targeted gene. Alternatively, the genetic engineering to decrease protein expression may be accomplished using RNAi techniques, microRNA or antisense RNA to suppress expression of the target gene.

Once the targeted gene knockout or suppression of expression approach has been carried out, the resulting population of cells may be screened to select and enrich for those cells exhibiting the phenotype of interest, for example decreased expression of surface-exposed MHC-I. Suitable techniques for screening and enrichment are known in the art and include flow cytometry and fluorescence-activated cell sorting (FACS).

In some embodiments, the producer cell comprises a genetically engineered disruption of a gene encoding P2-microglobulin. P2-microglobulin stabilises MHC-I, thus cells deficient in P2-microglobulin will exhibit decreased expression of MHC-1 on the surface of the cell. The cell may comprise genetically engineered disruptions in all copies of the gene encoding P2-microglobulin.

In another embodiment, the cell comprises a genetically engineered disruption of one or more gene encoding an MHC-I α chain. The cell may comprise genetically engineered disruptions in all copies of the gene encoding an MHC-I α chain.

The cell may comprise both genetically engineered disruptions of genes encoding P2-microglobulin and genetically engineered disruptions of genes encoding an MHC-I α chain.

Decreased expression of MHC-I on the surface of the cell may refer to a decrease in the number of MHC-I molecules that are expressed on the surface of the cell that has been genetically engineered, in comparison to the number of MHC-I molecules that are expressed on the surface of a cell lacking the genetic engineering, but under otherwise substantially identical conditions. The expression of MHC-I on the surface of the cell may be decreased such that the number of surface-exposed MHC-I molecules is, for example, less than about 50%, 40%, 30%, 25%, 20%, 15%, 10%, 5%, 4%, 3%, 2% or 1% of the number of surface-exposed MHC-I molecules that are displayed in the absence of the genetic engineering. In some embodiments, the expression of MHC-I on the surface of the cell is decreased such that the number of surface-exposed MHC-I molecules is 0% of the number of surface-exposed MHC-I molecules that are displayed in the absence of the genetic engineering.

The expression of MHC-I on the surface of the cell is preferably decreased such that the cell is substantially devoid of surface-exposed MHC-I molecules. In this context, “substantially devoid” may mean that there is a substantial decrease in the number of MHC-I molecules that are expressed on the surface of the cell that has been genetically engineered, in comparison to the number of MHC-I molecules that are expressed on the surface of a cell lacking the genetic engineering, such that the immune response to MHC-I on lentiviral particles produced by the cell is decreased to a therapeutically useful degree.

Suitably, the lentiviral particle of the present invention has a lower concentration of MHC-I molecules on its surface than a lentiviral particle obtained from an unmodified producer cell. Suitably, the lentiviral particle has less than about 50%, 40%, 30%, 25%, 20%, 15%, 10%, 5%, 4%, 3%, 2% or 1% of the number of surface-exposed MHC-I molecules that are displayed on a lentiviral particle obtained from an unmodified producer cell. In some embodiments, the lentiviral particle has less than about 20% of the number of surface-exposed MHC-I molecules that are displayed on a lentiviral particle obtained from an unmodified producer cell.

In some embodiments, the lentiviral particle of the present invention is substantially devoid of MHC-I molecules on its surface. In this context, “substantially devoid” may mean that there is no detectable immune response due to the molecules on the surface of the lentiviral particle. In some embodiments, the lentiviral particle of the present invention is free of MHC-1 molecules on its surface. In this context, “free” may mean that there are no detectable molecules (e.g. by immunostaining and electron microscopy) on the surface of the lentiviral particle. As used herein, “not detectable” may refer to levels which are not statistically significantly different compared to background levels.

In some embodiments, the lentiviral particle of the present invention has decreased HLA-A, HLA-B and/or HLA-C molecules on its surface. Suitably, the lentiviral particle has less than about 50%, 40%, 30%, 25%, 20%, 15%, 10%, 5%, 4%, 3%, 2% or 1% of the number of surface-exposed HLA-A molecules that are displayed on a lentiviral particle obtained from an unmodified producer cell. Suitably, the lentiviral particle has less than about 50%, 40%, 30%, 25%, 20%, 15%, 10%, 5%, 4%, 3%, 2% or 1% of the number of surface-exposed HLA-B molecules that are displayed on a lentiviral particle obtained from an unmodified producer cell.

Suitably, the lentiviral particle has less than about 50%, 40%, 30%, 25%, 20%, 15%, 10%, 5%, 4%, 3%, 2% or 1% of the number of surface-exposed HLA-C molecules that are displayed on a lentiviral particle obtained from an unmodified producer cell.

In some embodiments, the lentiviral particle of the present invention is substantially devoid of HLA-A, HLA-B and/or HLA-C molecules on its surface. In some embodiments, the lentiviral particle of the present invention is substantially devoid of HLA-A, HLA-B and HLA-C molecules on its surface. In some embodiments, the lentiviral particle of the present invention is free of HLA-A, HLA-B and/or HLA-C molecules on its surface. In some embodiments, the lentiviral particle of the present invention is free of HLA-A, HLA-B and HLA-C molecules on its surface.

As described above, an HLA-I molecule consists of two polypeptide chains, an HLA-I heavy chain (a chain) and P2 microglobulin (P2M or p chain). The HLA-I α chain and P2M are linked non-covalently.

The skilled person would readily be able to determine amino acid and nucleic acid sequences of HLA-I α chains. For example, the HLA-I α chains may be identified in a genome sequence using their location within the major histocompatibility complex region of the chromosome (see, for example, Penn et al. (2005) Major histocompatibility complex (MHC). eLS).

HLA-A alpha chains may have an amino acid sequence of UniProtKB P04439. Exemplary HLA-A alpha chains are provided by SEQ ID NOs: 16 and 17. Suitably, an HLA-A alpha chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 16 or 17. Suitably, a HLA-A alpha chain comprises or consists of the amino acid sequence of SEQ ID NO: 16 or 17.

(SEQ ID NO: 16)
MAVMAPRTLLLLLSGALALTQTWAGSHSMRYFFTSVSRPGRGEPRFIAVG
YVDDTQFVREDSDAASQRMEPRAPWIEQEGPEYWDQETRNVKAQSQTDRV
DLGTLRGYYNQSEAGSHTIQIMYGCDVGSDGRFLRGYRQDAYDGKDYIAL
NEDLRSWTAADMAAQITKRKWEAAHEAEQLRAYLDGTCVEWLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIPIVGIIAGLVLLGAVITGAVVAAVMWRRKSSDRKGGSYTQAASS
DSAQGSDVSLTACKV
(SEQ ID NO: 17)
MAVMAPRTLLLLLSGALALTQTWAGSHSMRYFFTSVSRPGRGEPRFIAVG
YVDDTQFVREDSDAASQRMEPRAPWIEQEGPEYWDQETRNVKAQSQTDRV
DLGTLRGYYNQSEAGSHTIQIMYGCDVGSDGRFLRGYRQDAYDGKDYIAL
NEDLRSWTAADMAAQITKRKWEAAHAAEQQRAYLEGRCVEWLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIPIVGIIAGLVLLGAVITGAVVAAVMWRRKSSGGEGVKDRKGGSY
TQAASSDSAQGSDVSLTACKV

HLA-B alpha chains may have an amino acid sequence of UniProtKB P01889. An exemplary HLA-B alpha chain is provided by SEQ ID NO: 18. Suitably, an HLA-B alpha chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 18. Suitably, a HLA-B alpha chain comprises or consists of the amino acid sequence of SEQ ID NO: 18.

(SEQ ID NO: 18)
MLVMAPRTVLLLLSAALALTETWAGSHSMRYFYTSVSRPGRGEPRFISVG
YVDDTQFVREDSDAASPREEPRAPWIEQEGPEYWDRNTQIYKAQAQTDRE
SLRNLRGYYNQSEAGSHTLQSMYGCDVGPDGRLLRGHDQYAYDGKDYIAL
NEDLRSWTAADTAAQITQRKWEAAREAEQRRAYLEGECVEWLRRYLENGK
DKLERADPPKTHVTHHPISDHEATLRCWALGFYPAEITLTWQRDGEDQTQ
DTELVETRPAGDRTFQKWAAVVVPSGEEQRYTCHVQHEGLPKPLTLRWEP
SSQSTVPIVGIVAGLAVLAVVVIGAVVAAVMCRRKSSGGKGGSYSQAACS
DSAQGSDVSLTA

HLA-C alpha chains may have an amino acid sequence of UniProtKB P10321. Exemplary HLA-C alpha chains are provided by SEQ ID NOs: 19 and 20. Suitably, an HLA-C alpha chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 19 or 20. Suitably, a HLA-C alpha chain comprises or consists of the amino acid sequence of SEQ ID NO: 19 or 20.

(SEQ ID NO: 19)
MRVMAPRALLLLLSGGLALTETWACSHSMRYFDTAVSRPGRGEPRFIS
VGYVDDTQFVREDSDAASPRGEPRAPWVEQEGPEYWDRETQKYKRQAQAD
RVSLRNLRGYYNQSEDGSHTLQRMSGCDLGPDGRLLRGYDQSAYDGKDYI
ALNEDLRSWTAADTAAQITQRKLEAARAAEQLRAYLEGTCVEWLRRYLEN
GKETLQRAEPPKTHVTHHPLSDHEATLRCWALGFYPAEITLTWQRDGEDQ
TQDTELVETRPAGDGTFQKWAAVVVPSGQEQRYTCHMQHEGLQEPLTLSW
EPSSQPTIPIMGIVAGLAVLVVLAVLGAVVTAMMCRRKSSGGKGGSCSQA
ACSNSAQGSDESLITCKA
(SEQ ID NO: 20)
MRVMAPRALLLLLSGGLALTETWACSHSMRYEDTAVSRPGRGEPRFISVG
YVDDTQFVREDSDAASPRGEPRAPWVEQEGPEYWDRETQKYKRQAQADRV
SLRNLRGYYNQSEDGSHTLQRMSGCDLGPDGRLLRGYDQSAYDGKDYIAL
NEHLRSCTAADTAAQITQRKLEAARAAEQLRAYLEGTCVEWLRRYLENGK
ETLQRAEPPKTHVTHHPLSDHEATLRCWALGFYPAEITLTWQRDGEDQTQ
DTELVETRPAGDGTFQKWAAVVVPSGQEQRYTCHMQHEGLQEPLTLRWGG
KGGSCSQAACSNSAQGSDESLITCKA

Amino acid and nucleic acid sequences encoding P2M are also known in the art. For example, a nucleic acid sequence of a human P2M is deposited as GenBank Accession No. NM_004048.

An HLA p chain may be that of UniProtKB P61769. An exemplary HLA p chain is provided by SEQ ID NO: 21. Suitably, an HLA p chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 21. Suitably, an HLA p chain comprises or consists of the amino acid sequence of SEQ ID NO: 21.

(SEQ ID NO: 21)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM

The lentiviral particle of the present invention may be a CD47^{high}/MHC-I^{free} lentiviral particle or a CD47^{high}/MHC-I^{low} lentiviral particle. In preferred embodiments, the lentiviral particle of the present invention is a CD47^{high}/MHC-I^{free} lentiviral particle.

The lentiviral particle of the present invention may be obtained from a CD47^{high}/MHC-I^{free} producer cell or a CD47^{high}/MHC-I^{low} producer cell. In preferred embodiments, the lentiviral particle of the present invention is obtained from a CD47^{high}/MHC-I^{free} producer cell.

### Enveloped Virus-Like Particles (VLPs)

Virus-like particles (VLPs) closely resemble viruses, but do not comprise viral genetic material. VLPs may be synthesised through expression of viral structural proteins, which can then self assemble into the virus-like structure. VLPs may retain the ability to transduce cells and release cargo. Disclosures herein in relation to enveloped viral particles may also apply to enveloped VLPs where appropriate in the context.

VLPs may be derived from enveloped viruses, such as lentiviruses.

VLPs may be used for the delivery of protein cargo, such as Cas9 nuclease or ribonucleoproteins (RNPs). VLPs may be used for the delivery of one or more component of gene editing machinery (e.g. CRISPR/Cas9, base editor or prime editor).

### Regulatory Elements

The enveloped viral particle (e.g. lentiviral particle) of the present invention may further comprise one or more regulatory elements which may act pre- or post-transcriptionally. Suitably, the protein-coding sequence is operably linked to one or more regulatory elements which may act pre- or post-transcriptionally. The one or more regulatory elements may facilitate expression of the protein in liver cells (e.g. hepatocytes).

As used herein, a “regulatory element” may refer any nucleotide sequence that facilitates expression of a polypeptide, for example acts to increase expression of a transcript or to enhance mRNA stability. Suitable regulatory elements include for example promoters, enhancer elements, post-transcriptional regulatory elements, polyadenylation sites and Kozak sequences.

### Promoter

The enveloped viral particle (e.g. lentiviral particle) of the present invention may comprise a promoter, such as a liver-specific (e.g. hepatocyte-specific) promoter. Suitably, the protein-coding sequence is operably linked to a promoter, such as a liver-specific (e.g. hepatocyte-specific) promoter.

A “promoter” may refer to a region of DNA that leads to initiation of transcription of a gene. Promoters are located near the transcription start sites of genes.

As used herein, a “tissue-specific promoter” may refer to a promoter which preferentially facilitates expression of a transgene in a specific type of cells or tissue. Suitably, a tissue-specific promoter may facilitate higher expression of a transgene in one cell type as compared to other cell types. Higher expression may be measured for example by measuring the expression of a transgene, for example green fluorescence protein (GFP), operably linked to the promoter, wherein expression of the transgene correlates with the ability of the promoter to facilitate expression of a gene. For example, a tissue-specific promoter may be a promoter which facilitates transgene expression levels at least 10% higher, at least 20% higher, at least 30% higher, at least 40% higher, at least 50% higher, at least 100% higher, at least 200% higher, at least 300% higher, at least 400% higher, at least 500% higher or at least 1000% higher in one cell type as compared to expression levels in other cell types.

In some embodiments, the promoter is a liver-specific promoter. In some embodiments, the promoter is a hepatocyte-specific promoter.

Suitably, the promoter may be (or may be derived from) a promoter associated with a gene with selective expression in human liver cells (e.g. hepatocytes). Suitably, the promoter may be (or may be derived from) a promoter associated with a gene with selective expression in human hepatocyte cells. Methods to identify promoters associated with genes will be well known to the skilled person.

Exemplary liver-specific and/or hepatocyte-specific promoters are described in Kattenhorn, et al. (2016) Human Gene Therapy 27: 947-961 and include transthyretin (TTR) promoters, alpha-1-antityrpsin (AAT) promoters, thyroxine-binding globulin (TBG) promoters, APoE/hAAT promoters, HCR-hAAT promoters, LP1 promoters and HLP promoters.

An engineered promoter variant derived from any of these promoters may be used, provided that the variant retains the capacity to drive liver-specific and/or hepatocyte-specific expression of a transgene which is operably coupled to the promoter. A skilled person will be able to arrive at such variants using methods known in the art. The variant may, for example, have at least 70%, at least 80%, at least 90%, at least 95%, at least 96%, at least 97%, at least 98% or at least 99% sequence identity to any of the promoters.

A fragment of any of these promoters (or variants thereof) may be used, provided that the fragment retains the capacity to drive liver-specific and/or hepatocyte-specific expression of a transgene which is operably coupled to the promoter. A skilled person will be able to arrive at such fragments using methods known in the art. The fragment may be, for example, at least 200 nucleotides, at least 300 nucleotides, at least 400 nucleotides, at least 500 nucleotides or at least 1000 nucleotides in length.

In some embodiments, the promoter is selected from the group consisting of: a transthyretin (TTR) promoter, an alpha-1-antityrpsin (AAT) promoter, a thyroxine-binding globulin (TBG) promoter, an APoE/hAAT promoter, a HCR-hAAT promoter, a LP1 promoter and a HLP promoter.

In some embodiments, the promoter is a TTR promoter, or a variant and/or fragment thereof. In some embodiments, the promoter is an enhanced TTR (ET) promoter, or a variant and/or fragment thereof.

The promoter may be a constitutive promoter. As used herein, a “constitutive promoter” is a promoter which is always active.

Alternatively, the promoter may be an inducible promoter. As used herein, an “inducible promoter” is a promoter which is only active under specific conditions. For example, expression of the transgene may be induced by a small molecule or drug (e.g. which binds to a promoter, regulatory sequence or to a transcriptional repressor or activator molecule) or by using an environmental trigger. Types of inducible promoter include chemically-inducible promoters (e.g. a Tet-on system); temperature-inducible promoters (e.g. Hsp70 or Hsp90-derived promoters); and light-inducible promoters. Suitably, the promoter is chemically-inducible. Any suitable method for engineering an inducible promoter may be used.

### Enhancer Elements

The enveloped viral particle (e.g. lentiviral particle) of the present invention may comprise an enhancer, such as a liver-specific (e.g. hepatocyte-specific) enhancer. Suitably, the protein-coding sequence is operably linked to an enhancer, such as a liver-specific (e.g. hepatocyte-specific) enhancer.

An “enhancer” or “enhancer element” may refer a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site.

As used herein, a “tissue-specific enhancer” is an enhancer which preferentially facilitates expression of a gene in specific cells or tissues. Suitably, a tissue-specific enhancer may facilitate higher expression of a gene in specific cells types as compared to other cell types. Higher expression may be measured for example by measuring the expression of a transgene, for example green fluorescence protein (GFP), operably linked to the enhancer, wherein expression of the transgene correlates with the ability of the enhancer to facilitate expression of a gene. For example, a tissue-specific enhancer may be an enhancer which facilitates gene expression levels at least 10% higher, at least 20% higher, at least 30% higher, at least 40% higher, at least 50% higher, at least 100% higher, at least 200% higher, at least 300% higher, at least 400% higher, at least 500% higher or at least 1000% higher in a specific cell-type compared to expression levels in other cell types.

Suitable tissue-specific enhancers will be well known to the skilled person. The enhancer may be a liver-specific enhancer, preferably a hepatocyte-specific enhancer.

Suitably, the enhancer may be (or may be derived from) an enhancer associated with a gene with selective expression in human liver cells (e.g. hepatocytes). Suitably, the enhancer may be (or may be derived from) an enhancer associated with a gene with selective expression in human hepatocyte cells. Methods to identify the enhancer regions associated with genes will be well known to the skilled person.

Exemplary liver-specific and/or hepatocyte-specific enhancers are described in Kramer et al. (2003) Molecular Therapy 7: 375-385, and include enhancer regions of the albumin, α1-antitrypsin, hepatitis B virus core protein, and hemopexin genes. Other liver-specific and/or hepatocyte-specific enhancers include apolipoprotein E (APoE) enhancers, hepatic control region (HCR) enhancers and alpha-1-antitrypsin (AAT) enhancers.

An engineered enhancer variant derived from any of these enhancers may be used, provided that the variant retains the capacity to drive liver-specific and/or hepatocyte-specific expression of a transgene which is operably coupled to the enhancer. A skilled person will be arrive at such variants using methods known in the art. The variant may have at least 70%, at least 80%, at least 90%, at least 95%, at least 96%, at least 97%, at least 98% or at least 99% sequence identity to any of the enhancers.

A fragment of any of these enhancers (or variants thereof) may be used, provided that the fragment retains the capacity to drive liver-specific and/or hepatocyte-specific expression of a transgene which is operably coupled to the enhancer. A skilled person will be able to arrive at such fragments using methods known in the art. The fragment may be at least 200 nucleotides, at least 300 nucleotides, at least 400 nucleotides, at least 500 nucleotides or at least 1000 nucleotides in length.

The enveloped viral particle (e.g. lentiviral particle) of the present invention may comprise a liver-specific promoter and/or a liver-specific enhancer. Suitably, the protein-coding sequence is operably linked to a liver-specific promoter and/or enhancer. Suitably, the protein-coding sequence is operably linked to a hepatocyte-specific promoter and/or enhancer. The promoter and enhancer may be a combination of any of the above, for example a hAAT promoter and an ApoE or HCR enhancer.

### Post-Transcriptional Regulatory Elements

The enveloped viral particle (e.g. lentiviral particle) of the present invention may comprise one or more further post-transcriptional regulatory elements (e.g. in addition to one or more miRNA target sequence). Suitably, the protein-coding sequence is operably linked to one or more further post-transcriptional regulatory elements. The further post-transcriptional regulatory element may improve gene expression.

The enveloped viral particle (e.g. lentiviral particle) of the present invention may comprise a Woodchuck Hepatitis Virus Post-transcriptional Regulatory Element (WPRE). Suitably, the protein-coding sequence is operably linked to a WPRE.

Suitable WPRE sequences will be well known to those of skill in the art (see, for example, Zufferey et al. (1999) Journal of Virology 73: 2886-2892; Zanta-Boussif et al. (2009) Gene Therapy 16: 605-619). Suitably, the WPRE is a wild-type WPRE or is a mutant WPRE. For example, the WPRE may be mutated to abrogate translation of the woodchuck hepatitis virus X protein (WHX), for example by mutating the WHX ORF translation start codon.

In some embodiments, the WPRE comprises or consists of a nucleotide sequence that has at least 70% sequence to SEQ ID NO: 22 or a fragment thereof. Suitably, the WPRE comprises or consists of a nucleotide sequence that has at least 75%, at least 80%, at least 85%, at least 90%, at least 95%, at least 96%, at least 97%, at least 98% or at least 99% sequence identity to SEQ ID NO: 22 or a fragment thereof.

In some embodiments, the WPRE comprises or consists of the nucleotide sequence SEQ ID NO: 22 or a fragment thereof.

(SEQ ID NO: 22)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGG
ACGTCCTTCTGCTACGTCCCTTCGGCCCTCAATCCAGCGGACCTTCCTTC
CCGC

### Polyadenylation Sequence

The enveloped viral particle (e.g. lentiviral particle) of the present invention may comprise a polyadenylation sequence. Suitably, the protein-coding sequence is operably linked to a polyadenylation sequence. A polyadenylation sequence may be inserted after the protein-coding sequence to improve transgene expression.

A polyadenylation sequence typically comprises a polyadenylation signal, a polyadenylation site and a downstream element: the polyadenylation signal comprises the sequence motif recognised by the RNA cleavage complex; the polyadenylation site is the site of cleavage at which a poly-A tails is added to the mRNA; the downstream element is a GT-rich region which usually lies just downstream of the polyadenylation site, which is important for efficient processing.

Suitable polyadenylation sequences will be well known to those of skill in the art (see, for example, Schambach et al. (2007) Molecular Therapy 15: 1167-1173; Choi et al. (2014) Molecular Brain 7: 1-10). Exemplary polyadenylation sequences include the bGH poly(A) signal sequence and SV40 pA signal sequence.

### Kozak Sequence

The enveloped viral particle (e.g. lentiviral particle) of the present invention may comprise a Kozak sequence. Suitably, the protein-coding sequence is operably linked to a Kozak sequence. A Kozak sequence may be inserted before the start codon to improve the initiation of translation.

Suitable Kozak sequences will be well known to the skilled person (see, for example, Kozak (1987) Nucleic Acids Research 15: 8125-8148).

In some embodiments, the Kozak sequence comprises or consists of a nucleotide sequence that has at least 80% sequence identity to SEQ ID NO: 23 or a fragment thereof.

In some embodiments, the Kozak sequence comprises or consists of the nucleotide sequence SEQ ID NO: 23 or a fragment thereof.

(SEQ ID NO: 23)
GCCACC

### Other Cis-Acting Elements

The enveloped viral particle (e.g. lentiviral particle) of the present invention may comprise any other suitable cis-acting elements, such as one or more of a rev response element (RRE); a retroviral psi packaging element; a primer binding site (PBS); a TAT activation region (TAR); splice donor and acceptor sites; and central and terminal polypurine tracts.

### Long Terminal Repeats (LTRs)

The enveloped viral particle (e.g. lentiviral particle) of the present invention may comprise one or more long terminal repeat (LTR). LTRs are responsible for proviral integration and transcription. Typically, a naturally occurring LTR comprises U3, R, and U5 regions.

The enveloped viral particle (e.g. lentiviral particle) may comprise a 5′ LTR and/or a 3′ LTR. The enveloped viral particle (e.g. lentiviral particle) may comprise a 5′ LTR and a 3′ LTR. Suitably, a 5′ LTR comprises R and U5 regions, and optionally comprises a U3 region. Suitably, a 3′ LTR comprises U3, R and U5 regions.

Suitable LTR sequences will be well known to the skilled person (see, for example, Frech et al. (1996) Virology 224: 256-267).

In some embodiments, a LTR comprises or consists of a nucleotide sequence that has at least 70% sequence identity to SEQ ID NO: 24 or a fragment thereof. Suitably, a LTR comprises or consists of a nucleotide sequence that has at least 75%, at least 80%, at least 85%, at least 90%, at least 95%, at least 96%, at least 97%, at least 98% or at least 99% sequence identity to SEQ ID NO: 24 or a fragment thereof.

In some embodiments, a LTR comprises or consists of the nucleotide sequence SEQ ID NO: 24 or a fragment thereof.

(SEQ ID NO: 24)
TGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTC
AAGTAGTGTGTGCCCGTCTGTTGTGTGACTCTGGTAACTAGAGATCCCTC
AGACCCTTTTAGTCAGTGTGGAAAATCTCTAGCAG

The enveloped viral particle (e.g. lentiviral particle) of the present invention may comprise one or more self-inactivating long terminal repeat (SIN-LTR). A “SIN-LTR” may comprise a deletion that abolishes transcription of the full-length virus after it has incorporated into a host cell. For example, a 3′ SIN-LTR may comprise a deletion in the U3 region removing the promoter/enhancer elements (see, for example, Zufferey et al. (1998) Journal of Virology 72: 9873-9880). This deletion is copied into the 5′ LTR after reverse transcription, thereby making the gene expression in target cells dependent on an internal promoter of choice.

Suitable SIN-LTR sequences will be well known to the skilled person (see, for example, Zufferey et al. (1998) Journal of Virology 72: 9873-9880; Miyoshi et al. (1998) Journal of Virology 72: 8150-8157).

In some embodiments, the 5′ LTR comprises or consists of a nucleotide sequence that has at least 70% sequence identity to SEQ ID NO: 25 or a fragment thereof. Suitably, the 5′ LTR comprises or consists of a nucleotide sequence that has at least 75%, at least 80%, at least 85%, at least 90%, at least 95%, at least 96%, at least 97%, at least 98% or at least 99% sequence identity to SEQ ID NO: 25 or a fragment thereof.

In some embodiments, the 5′ LTR comprises or consists of the nucleotide sequence SEQ ID NO: 25 or a fragment thereof.

(SEQ ID NO: 25)
GGGTCTCTCTGGTTAGACCAGATCTGAGCCTGGGAGCTCTCTGGCTAACT
AGGGAACCCACTGCTTAAGCCTCAATAAAGCTTGCCTTGAGTGCTTCAAG
TAGTGTGTGCCCGTCTGTTGTGTGACTCTGGTAACTAGAGATCCCTCAGA
CCCTTTTAGTCAGTGTGGAAAATCTCTAGCAG

In some embodiments, the 5′ LTR and/or the 3′ LTR comprises or consists of a nucleotide sequence that has at least 70% sequence identity to SEQ ID NO: 24 or a fragment thereof. Suitably, the 5′ LTR and/or the 3′ LTR comprises or consists of a nucleotide sequence that has at least 75%, at least 80%, at least 85%, at least 90%, at least 95%, at least 96%, at least 97%, at least 98% or at least 99% sequence identity to SEQ ID NO: 24 or a fragment thereof.

In some embodiments, the 5′ LTR and/or the 3′ LTR comprises or consists of the nucleotide sequence SEQ ID NO: 24 or a fragment thereof.

In some embodiments, the 5′ LTR and the 3′ LTR comprise or consist of a nucleotide sequence that has at least 70% sequence identity to SEQ ID NO: 24 or a fragment thereof. Suitably, the 5′ LTR and the 3′ LTR comprise or consist of a nucleotide sequence that has at least 75%, at least 80%, at least 85%, at least 90%, at least 95%, at least 96%, at least 97%, at least 98% or at least 99% sequence identity to SEQ ID NO: 24 or a fragment thereof.

In some embodiments, the 5′ LTR and the 3′ LTR comprise or consist of the nucleotide sequence SEQ ID NO: 24 or a fragment thereof.

### Primer Binding Site (PBS)

The enveloped viral particle (e.g. lentiviral particle) of the present invention may comprise a primer binding site (PBS). A PBS is a cis-acting element where a primer may bind to initiate reverse transcription of the RNA genome (see, for example, Lanchy et al. (1998) Journal of Biological Chemistry 273: 24425-24432).

Suitable retroviral PBSs will be well known to the skilled person.

In some embodiments, a PBS comprises or consists of a nucleotide sequence that has at least 70% sequence identity to SEQ ID NO: 26 or a fragment thereof. Suitably, a PBS comprises or consists of a nucleotide sequence that has at least 75%, at least 80%, at least 85%, at least 90%, at least 95%, at least 96%, at least 97%, at least 98% or at least 99% identical to SEQ ID NO: 26 or a fragment thereof.

In some embodiments, a PBS comprises or consists of the nucleotide sequence SEQ ID NO: 26 or a fragment thereof.

(SEQ ID NO: 26)
TGGCGCCCGAACAGGGACTTGAAAGCGAAAGGGAAACCAGAGGAGCTCTC
TCGACGCAGGACTCGGCTTGCTGAAGCGCGCACGGCAAGAGGCGAGGGGC
GGCGACTGGTGAGTACGCCAAAAATTTTGACTAGCGGAGGCTAGAAGGAG
AGAG

Retroviral Psi Packaging Element The enveloped viral particle (e.g. lentiviral particle) of the present invention may comprise a retroviral psi packaging element. A retroviral psi packaging element is a cis-acting element which is involved in regulating the process of packaging the retroviral RNA genome into the viral capsid during replication (see, for example, McBride et al. (1997) Journal of Virology 71: 4544-4554). A retroviral psi packaging element may form part of the 5′ region of the gag gene.

Suitable retroviral psi packaging elements will be well known to the skilled person.

In some embodiments, a retroviral psi packaging element comprises or consists of a nucleotide sequence that has at least 70% sequence identity to SEQ ID NO: 27 or a fragment thereof. Suitably, a retroviral psi packaging element comprises or consists of a nucleotide sequence that has at least 75%, at least 80%, at least 85%, at least 90%, at least 95%, at least 96%, at least 97%, at least 98% or at least 99% sequence identity to SEQ ID NO: 27 or a fragment thereof.

In some embodiments, a retroviral psi packaging element comprises or consists of the nucleotide sequence SEQ ID NO: 27 or a fragment thereof.

(SEQ ID NO: 27)
ATGGGTGCGAGAGCGTCAGTATTAAGCGGGGGAGAATTAGATCGCGATGG
GAAAAAATTCGGTTAAGGCCAGGGGGAAAGAAAAAATATAAATTAAAACA
TATAGTATGGGCAAGCAGGGAGCTAGAACGATTCGCAGTTAATCCTGGCC
TGTTAGAAACATCAGAAGGCTGTAGACAAATACTGGGACAGCTACAACCA
TCCCTTCAGACAGGATCAGAAGAACTTAGATCATTATATAATACAGTAGC
AACCCTCTATTGTGTGCATCAAAGGATAGAGATAAAAGACACCAAGGAAG
CTTTAGACAAGATAGAGGAAGAGCAAAACAAAAGTAAGACCACCGCACAG
CAAGCGGCCGCTGAT

### Rev Response Element (RRE)

The enveloped viral particle (e.g. lentiviral particle) of the present invention may comprise a rev response element (RRE). A RRE is a cis-acting element that enables the efficient export of RNA transcripts of the integrated provirus from the nucleus to the cytoplasm of an infected target cell (see, for example, Pollard et al. (1998) Annual Review of Microbiology 52: 491-532).

Suitable RRE sequences will be well known to the skilled person.

In some embodiments, a RRE comprises or consists of a nucleotide sequence that has at least 70% sequence identity to SEQ ID NO: 28 or a fragment thereof. Suitably, a RRE comprises or consists of a nucleotide sequence that has at least 75%, at least 80%, at least 85%, at least 90%, at least 95%, at least 96%, at least 97%, at least 98% or at least 99% identical to SEQ ID NO: 28 or a fragment thereof.

In some embodiments, a RRE comprises or consists of the nucleotide sequence SEQ ID NO: 28 or a fragment thereof.

(SEQ ID NO: 28)
GGAGCTTTGTTCCTTGGGTTCTTGGGAGCAGCAGGAAGCACTATGGGCGC
AGCGTCAATGACGCTGACGGTACAGGCCAGACAATTATTGTCTGGTATAG
TGCAGCAGCAGAACAATTTGCTGAGGGCTATTGAGGCGCAACAGCATCTG
TTGCAACTCACAGTCTGGGGCATCAAGCAGCTCCAGGCAAGAATCCTGGC
TGTGGAAAGATACCTAAAGGATCAACAGCTCCTGGGGATTT

Central Polypurine Tract (cPPT)

The enveloped viral particle (e.g. lentiviral particle) of the present invention may comprise a central polypurine tract (cPPT). A cPPT may allow initiation of plus-strand synthesis (see, for example, Follenzi et al. (2000) Nature Genetics 25: 217-222).

Suitable cPPT sequences will be well known to the skilled person.

In some embodiments, a cPPT comprises or consists of a nucleotide sequence that has at least 70% sequence identity to SEQ ID NO: 29 or a fragment thereof. Suitably, a cPPT comprises or consists of a nucleotide sequence that has at least 75%, at least 80%, at least 85%, at least 90%, at least 95%, at least 96%, at least 97%, at least 98% or at least 99% sequence identity to SEQ ID NO: 29 or a fragment thereof.

In some embodiments, a cPPT comprises or consists of the nucleotide sequence SEQ ID NO: 29 or a fragment thereof.

(SEQ ID NO: 29)
AACTTTTAAAAGAAAAGGGGGGATTGGGGGGTACAGTGCAGGGGAAAGAA
TAGTAGACATAATAGCAACAGACATACAAACTAAAGAATTACAAAAACAA
ATTACAAAAATTCAAAATTTTATC

### Nucleotide of Interest

The viral particles of the invention may comprise a transgene, for example encoding a protein of interest. Preferably, the transgene gives rise to a therapeutic effect.

Suitable transgenes include, but are not limited to sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, ribozymes, miRNA target sequences, a transdomain negative mutant of a target protein, toxins, conditional toxins, antigens, tumour suppressor proteins, growth factors, transcription factors, membrane proteins, surface receptors, anti-cancer molecules, vasoactive proteins and peptides, anti-viral proteins and ribozymes, and derivatives thereof (such as derivatives with an associated reporter group). The transgene may also encode pro-drug activating enzymes.

An example of a transgene is the coagulation Factor VIII or Factor IX or engineered derivatives thereof, which may be used for gene therapy of haemophilia or the beta-globin chain which may be used for gene therapy of thalassemia/sickle cell disease.

The transgene may encode, for example a chimeric antigen receptor (CAR). The transgene may encode LDLR. In some embodiments, the enveloped viral particle (e.g. lentiviral particle) comprises a nucleotide sequence encoding LDLR.

In some embodiments, the LDLR comprises or consists of an amino acid sequence that has at least 75%, 80%, 85%, 90%, 95%, 96%, 97%, 98% or 99% sequence identity to SEQ ID NO: 5, or a fragment thereof.

In some embodiments, the LDLR comprises or consists of the amino acid sequence of SEQ ID NO: 5, or a fragment thereof.

An example nucleotide sequence encoding LDLR is:

(SEQ ID NO: 33; human LDLR)
ATGGGGCCCTGGGGCTGGAAATTGCGCTGGACCGTCGCCTTGCTCCTCGCCGCGGCGGGGACTGCAGT
GGGCGACAGATGCGAAAGAAACGAGTTCCAGTGCCAAGACGGGAAATGCATCTCCTACAAGTGGGTCT
GCGATGGCAGCGCTGAGTGCCAGGATGGCTCTGATGAGTCCCAGGAGACGTGCTTGTCTGTCACCTGC
AAATCCGGGGACTTCAGCTGTGGGGGCCGTGTCAACCGCTGCATTCCTCAGTTCTGGAGGTGCGATGG
CCAAGTGGACTGCGACAACGGCTCAGACGAGCAAGGCTGTCCCCCCAAGACGTGCTCCCAGGACGAGT
TTCGCTGCCACGATGGGAAGTGCATCTCTCGGCAGTTCGTCTGTGACTCAGACCGGGACTGCTTGGAC
GGCTCAGACGAGGCCTCCTGCCCGGTGCTCACCTGTGGTCCCGCCAGCTTCCAGTGCAACAGCTCCAC
CTGCATCCCCCAGCTGTGGGCCTGCGACAACGACCCCGACTGCGAAGATGGCTCGGATGAGTGGCCGC
AGCGCTGTAGGGGTCTTTACGTGTTCCAAGGGGACAGTAGCCCCTGCTCGGCCTTCGAGTTCCACTGC
CTAAGTGGCGAGTGCATCCACTCCAGCTGGCGCTGTGATGGTGGCCCCGACTGCAAGGACAAATCTGA
CGAGGAAAACTGCGCTGTGGCCACCTGTCGCCCTGACGAATTCCAGTGCTCTGATGGAAACTGCATCC
ATGGCAGCCGGCAGTGTGACCGGGAATATGACTGCAAGGACATGAGCGATGAAGTTGGCTGCGTTAAT
GTGACACTCTGCGAGGGACCCAACAAGTTCAAGTGTCACAGCGGCGAATGCATCACCCTGGACAAAGT
CTGCAACATGGCTAGAGACTGCCGGGACTGGTCAGATGAACCCATCAAAGAGTGCGGGACCAACGAAT
GCTTGGACAACAACGGCGGCTGTTCCCACGTCTGCAATGACCTTAAGATCGGCTACGAGTGCCTGTGC
CCCGACGGCTTCCAGCTGGTGGCCCAGCGAAGATGCGAAGATATCGATGAGTGTCAGGATCCCGACAC
CTGCAGCCAGCTCTGCGTGAACCTGGAGGGTGGCTACAAGTGCCAGTGTGAGGAAGGCTTCCAGCTGG
ACCCCCACACGAAGGCCTGCAAGGCTGTGGGCTCCATCGCCTACCTCTTCTTCACCAACCGGCACGAG
GTCAGGAAGATGACGCTGGACCGGAGCGAGTACACCAGCCTCATCCCCAACCTGAGGAACGTGGTCGC
TCTGGACACGGAGGTGGCCAGCAATAGAATCTACTGGTCTGACCTGTCCCAGAGAATGATCTGCAGCA
CCCAGCTTGACAGAGCCCACGGCGTCTCTTCCTATGACACCGTCATCAGCAGAGACATCCAGGCCCCC
GACGGGCTGGCTGTGGACTGGATCCACAGCAACATCTACTGGACCGACTCTGTCCTGGGCACTGTCTC
TGTTGCGGATACCAAGGGCGTGAAGAGGAAAACGTTATTCAGGGAGAACGGCTCCAAGCCAAGGGCCA
TCGTGGTGGATCCTGTTCATGGCTTCATGTACTGGACTGACTGGGGAACTCCCGCCAAGATCAAGAAA
GGGGGCCTGAATGGTGTGGACATCTACTCGCTGGTGACTGAAAACATTCAGTGGCCCAATGGCATCAC
CCTAGATCTCCTCAGTGGCCGCCTCTACTGGGTTGACTCCAAACTTCACTCCATCTCAAGCATCGATG
TCAACGGGGGCAACCGGAAGACCATCTTGGAGGATGAAAAGAGGCTGGCCCACCCCTTCTCCTTGGCC
GTCTTTGAGGACAAAGTATTTTGGACAGATATCATCAACGAAGCCATTTTCAGTGCCAACCGCCTCAC
AGGTTCCGATGTCAACTTGTTGGCTGAAAACCTACTGTCCCCAGAGGATATGGTTCTCTTCCACAACC
TCACCCAGCCAAGAGGAGTGAACTGGTGTGAGAGGACCACCCTGAGCAATGGCGGCTGCCAGTATCTG
TGCCTCCCTGCCCCGCAGATCAACCCCCACTCGCCCAAGTTTACCTGCGCCTGCCCGGACGGCATGCT
GCTGGCCAGGGACATGAGGAGCTGCCTCACAGAGGCTGAGGCTGCAGTGGCCACCCAGGAGACATCCA
CCGTCAGGCTAAAGGTCAGCTCCACAGCCGTAAGGACACAGCACACAACCACCCGACCTGTTCCCGAC
ACCTCCCGGCTGCCTGGGGCCACCCCTGGGCTCACCACGGTGGAGATAGTGACAATGTCTCACCAAGC
TCTGGGCGACGTTGCTGGCAGAGGAAATGAGAAGAAGCCCAGTAGCGTGAGGGCTCTGTCCATTGTCC
TCCCCATCGTGCTCCTCGTCTTCCTTTGCCTGGGGGTCTTCCTTCTATGGAAGAACTGGCGGCTTAAG
AACATCAACAGCATCAACTTTGACAACCCCGTCTATCAGAAGACCACAGAGGATGAGGTCCACATTTG
CCACAACCAGGACGGCTACAGCTACCCCTCGAGACAGATGGTCAGTCTGGAGGATGACGTGGCGTGA

In some embodiments, the nucleotide sequence encoding LDLR comprises or consists of a nucleotide sequence that has at least 75%, 80%, 85%, 90%, 95%, 96%, 97%, 98% or 99% sequence identity to SEQ ID NO: 33, or a fragment thereof.

In some embodiments, the nucleotide sequence encoding LDLR comprises or consists of the nucleotide sequence of SEQ ID NO: 33, or a fragment thereof.

### Pharmaceutical Composition

The enveloped viral particles or transduced cells of the invention may be formulated for administration to subjects with a pharmaceutically acceptable carrier, diluent or excipient. The enveloped VLPs of the invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy products is preferably performed in compliance with FACT-JACIE International Standards for cellular therapy.

A pharmaceutical composition may be a composition that comprises or consists of a therapeutically effective amount of a pharmaceutically active agent (e.g. the enveloped viral particle). A pharmaceutical composition preferably includes a pharmaceutically acceptable carrier, diluent or excipient (including combinations thereof).

By “pharmaceutically acceptable” it is included that the formulation is sterile and pyrogen free. The carrier, diluent, and/or excipient must be “acceptable” in the sense of being compatible with the enveloped viral particle and not deleterious to the recipients thereof. Typically, the carriers, diluents and excipients will be saline or infusion media which will be sterile and pyrogen free, however other acceptable carriers, diluents and excipients may be used.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as (or in addition to) the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The enveloped viral particle, cell, or pharmaceutical composition according to the present invention may be administered in a manner appropriate for treating and/or preventing the diseases described herein. The enveloped VLP according to the present invention may be administered in a manner appropriate for treating and/or preventing the diseases described herein. Suitable administration routes will be known to the skilled person.

The quantity and frequency of administration may be determined by the skilled person, for example depending by such factors as the condition of the subject, and the type and severity of the subject's disease. The pharmaceutical composition may be formulated accordingly.

The enveloped viral particle, cell or pharmaceutical composition according to the present invention may be administered parenterally, (e.g. intravenous, intra-arterial, intramuscular, intrathecal, subcutaneous), or by infusion techniques. The enveloped viral particle, cell or pharmaceutical composition may be administered in the form of a sterile aqueous solution which may contain other substances, for example enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to the skilled person.

The enveloped viral particle, cell or pharmaceutical composition according to the present invention may be administered systemically, for example by intravenous injection or intraperitoneal injection. In some embodiments, the enveloped viral particle, cell or pharmaceutical composition according to the present invention is administered by intravenous injection. The pharmaceutical composition may be formulated accordingly.

The enveloped viral particle, cell or pharmaceutical composition according to the present invention may be administered locally, for example by direct injection, intra-arterial injection or intraportal injection. In some embodiments, the enveloped viral particle, cell or pharmaceutical composition according to the present invention is administered locally to the liver. In some embodiments, the enveloped viral particle, cell or pharmaceutical composition according to the present invention is administered by intrahepatic injection, intrahepatic arterial injection or intraportal injection. The pharmaceutical composition may be formulated accordingly.

The pharmaceutical compositions may comprise enveloped viral particles or cells of the invention in infusion media, for example sterile isotonic solution. The pharmaceutical composition may be enclosed in ampoules, disposable syringes or multiple dose vials made of glass or plastic.

The enveloped viral particle, cell or pharmaceutical composition may be administered in a single or in multiple doses. Suitably, the enveloped viral particle, cell or pharmaceutical composition may be administered in a single, one off dose. The pharmaceutical composition may be formulated accordingly.

The enveloped viral particle, cell or pharmaceutical composition may be administered at varying doses (e.g. measured in Transducing Units (TU) per kg). The physician in any event may determine the actual dosage which will be most suitable for any individual subject and the dosage may, for example, vary with the age, weight and response of the particular subject. The pharmaceutical composition may be formulated accordingly.

The enveloped viral particle, cell or pharmaceutical composition may be administered to any subject in need thereof. The subject may be a mammal (e.g. a human).

The enveloped viral particles or enveloped VLPs of the invention may be used to transduce cells ex vivo. The transduced cells may subsequently be administered to a subject.

In another aspect, the invention provides a method of transducing a cell, comprising contacting the cell with the enveloped viral particle or enveloped VLP of the invention. The method may be, for example, an in vitro or ex vivo method.

### Variants, Derivatives, Analogues and Fragments

In addition to the specific polypeptides and polynucleotides mentioned herein, the invention also encompasses variants, derivatives and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one or all of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one or all of its endogenous functions.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Polypeptides used in the invention may also have deletions, insertions or substitutions of amino acid residues which produce a silent change and result in a functionally equivalent polypeptide. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The effect of additions, deletions, substitutions, modifications, replacements and/or variations may be predicted using any suitable prediction tool, for example SIFT (Vaser et al. (2016) Nature Protocols 11: 1-9), PolyPhen-2 (Adzhubei et al. (2013) Current Protocols in Human Genetics 76: 7-20), CADD (Rentzsch et al. (2021) Genome Medicine 13: 1-12), REVEL (Ioannidis et al. (2016) The American Journal of Human Genetics 99: 877-885), MetaLR (Dong et al. (2015) Human Molecular Genetics 24: 2125-2137) and/or MutationAssessor (Reva et al. (2011) Nucleic Acids Research 39: e118-e118) or based on clinical data, for example ClinVar (Landrum et al. (2016) Nucleic Acids Research 44: D862-D868). Suitable additions, deletions, substitutions, modifications, replacements and/or variations may be considered tolerated, benign and/or likely benign.

Typically, a variant may have a certain identity with the wild type amino acid sequence or the wild type nucleotide sequence.

In the present context, a variant sequence is taken to include an amino acid sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs described herein refers to a sequence that has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (Devereux et al. (1984) Nucleic Acids Research 12: 387-395). Examples of other software that can perform sequence comparisons include, but are not limited to, the BLAST package (Altschul, et al. (1990) Journal of Molecular Biology 215: 403-410), BLAST 2 (Tatusova et al. (1999) FEMS Microbiology Letters 174: 247-250), FASTA (Pearson et al. (1988) PNAS 85: 2444-2448), EMBOSS Needle (Madeira et al. (2019) Nucleic Acids Research 47: W636-W641) and the GENEWORKS suite of comparison tools. For some applications, it is preferred to use EMBOSS Needle.

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives and fragments may be prepared using standard recombinant DNA techniques, such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded polypeptide. These methods are only illustrative of the numerous standard techniques known in the art for manipulation of DNA sequences and other known techniques may also be used.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

### Materials and Methods

### Plasmid Construction

Plasmids were generated using a combination of standard cloning techniques and gene synthesis. pMAX.coPCSK9, pMAX.coPCSK9.S127R plasmids were generated by exchanging the GFP sequence in plasmid pMAX.GFP (Amaxa) with respectively the codon-optimized (co) gene-synthesized PCSK9 and PCSK9.S127R (GenScript).

### Plasmid DNA Preparation

Large-scale preparation of plasmid DNA was carried out using the Macherey-Nagel endotoxin-free high purity plasmid maxi prep system, according to the manufacturer's instructions. After a modified alkaline/sodium dodecyl sulfate (SDS) procedure to lyse the cells and precipitate the genomic DNA (gDNA), the cleared lysate is passed over an ion-exchange column, at the appropriate salt concentration and pH. The plasmid DNA is eluted under high-salt conditions. It is then desalted and concentrated by alcohol precipitation. Plasmid DNA is finally resuspended in TE (10 mM TrisHcl pH 8.0, 1 mM ethylenediaminetetraacetic acid (EDTA)).

### LV Production

VSV.G-pseudotyped third-generation self-inactivating LV vectors were produced by calcium phosphate transient transfection into 293T cells. Nine million 293T cells were seeded 24 hours before transfection in 15-cm dishes. Two hours before transfection culture medium was replaced with 22.5 mL of fresh medium. For each dish, a solution containing a mix of the selected transfer plasmid, the packaging plasmids pMDLg/pRRE and pCMV.REV, pMD2.G and the pAdVantage (Promega) plasmid was prepared using 35, 12.5, 6.25, 9 and 15 μg of plasmid DNA, respectively. To produce LV with PCSK9 overexpression, plasmid pMAX.coPCSK9 or pMAX.coPCSK9.S127 was added to the mix, using respectively 7.5 and 3.75 μg of DNA. A 0.1×TE solution (10 mM Tris-HCl, 1 mM EDTA pH 8.0 in dH₂O) was added to the DNA mix to 1250 μL of final volume. The solution was left on a spinning wheel for 20-30 minutes. Meanwhile, 125 μL of CaCl₂ were aliquoted in tubes where mix would be sampled. Right before transfection, a precipitate was formed by adding 1250 μL of 2×HBS (281 mM NaCl, 100 mM HEPES, 1.5 mM Na₂HPO₄, pH 7.12) while the solution was kept in agitation on a vortex. The precipitate was immediately added to the culture medium and left on cells for 14-16 hours. After that the culture medium was changed with 16 mL of fresh one. The supernatant containing the vector was collected 54 hours after medium change. Subsequently supernatant was passed through a 0.22 μm filter (Millipore). Filtered supernatant was frozen at −80° C. (non-concentrated LV) or transferred into sterile 25×89 mm polyallomer tubes (Beckman) and centrifuged at 20,000 g for 120 min at 20° C. (Beckman Optima XL-100K Ultracentrifuge). Vector pellet was dissolved in the appropriate volume of phosphate-buffered saline (PBS) to allow a 500× concentration.

### LV Titration and Vector Copy Number (VCN) Determination

For LV titration, 1×10⁵ 293T cells were transduced with serial LV dilutions (1:103-107 for concentrated LV and 1:10¹-10⁵ for non-concentrated LV) in the presence of polybrene (8 μg/mL). For LV encoding for PGK.GFP, cells were analyzed by flow cytometry 3-7 days after transduction and infectious titer, expressed as transducing units (TU)/mL, was calculated using the formula TU/mL=(% GFP positive cells/100)×100,000× (1/dilution factor). For all other LV, gDNA was extracted 10 days after transduction, using Maxwell 16 Cell DNA Purification Kit (Promega), following the manufacturer's instructions. Vector copy number (VCN) was determined by digital droplet polymerase chain reaction (ddPCR) starting from 5-20 ng of template gDNA using primers (HIV fw: 5′-TACTGACGCTCTCGCACC-3′; HIV rv: 5′-TCTCGACGCAGGACTCG-3′) and a probe (FAM 5′-ATCTCTCTCCTTCTAGCCTC-3′) designed on the primer binding site region of LV. The amount of endogenous DNA was quantified by a primers/probe set designed on the human glyceraldehyde 3-phosphate dehydrogenase (GAPDH) gene (Applied Biosystems HS004831 11_cm) or on the TATA-box binding protein associated factor 7 (TAF7) gene (Applied Biosystems Rh02916247_s1). The PCR reaction was performed with each primer (900 nM) and the probe (250 nM) following the manufacturer's instructions (Bio-Rad), read with QX200 reader and analyzed with QuantaSoft software (Bio-Rad). VCN was calculated by the formula=(ng LV/ng endogenous DNA)×2. Each ddPCR run carried an internal control generated by using a CEM cell line stably carrying 6 vector integrations, which were previously determined by southern blot and fluorescent in situ hybridization (FISH) analysis. Infectious titer, expressed as TU/mL, was calculated using the formula TU/mL=VCN×100,000× (1/dilution factor). LV physical particles were measured by HIV-1 Gag p24 antigen immunocapture assay (Perkin Elmer) following the manufacturer's instructions. LV specific infectivity was calculated as the ratio between infectious titer and physical particles.

For LV VCN determination in cell lines and T cells, DNA was extracted using Maxwell 16 Cell DNA Purification Kit (Promega). Then VCN was quantified by ddPCR as already described for LV titration. For mouse experiments, DNA was extracted from whole liver samples using Maxwell 16 Tissue DNA Purification Kit (Promega). VCN in murine DNA was determined by ddPCR, starting from 5-20 ng of template gDNA using a primers/probe set designed on the primer binding site region of LV, as described for LV titration. The amount of endogenous murine DNA was quantified by a primers/probe set designed on the murine sema3a gene (Sema3A fw: 5′-ACCGATTCCAGATGATTGGC-3′; Sema3A rv: 5′-TCCATATTAATGCAGTGCTTGC-3′; Sema3A probe: HEX 5′-AGAGGCCTGTCCTGCAGCTCATGG-3′ BHQ1). The PCR reaction was performed with each primer (900 nM) and the probe (250 nM) following the manufacturer's instructions (Bio-Rad), read with QX200 reader and analyzed with QuantaSoft software (Bio-Rad).

### Cell Cultures

293T cells were maintained in Iscove's modified Dulbecco's medium (IMDM, Sigma) supplemented with 10% fetal bovine serum (FBS, Euroclone), penicillin and streptomycin 100 international units (IU)/mL (Lonza). Sub confluent cell lines were washed with sterile PBS (Sigma) and detached with 0.05% trypsin, 4 mM EDTA in PBS.

Primary human lymphocytes were magnetically sorted (Miltenyi Biotec) from buffy coats of three healthy donors. They were kept in culture at a concentration of 10⁶ cells/mL in IMDM supplemented with interleukin 7 (5 ng/ml), interleukin 15 (5 ng/ml), 10% FBS, 1% glutamine, penicillin 100 IU/ml and streptomycin 100 μg/ml. T lymphocytes were pre-stimulated in vitro with beads coated with αHuman-CD3 and αHuman-CD28 (3 beads/cell, Dynabeads human T-activator CD3/CD28; Invitrogen).

All cells were maintained in a 5% CO₂ humidified atmosphere at 37° C.

### Flow Cytometry

Flow cytometry analyses were performed using a FACSCanto analyzer (BD Biosciences), equipped with DIVA Software. Between 100,000-500,000 cells were harvested, washed with PBS or MACS buffer (PBS pH 7.2 0.5% bovine serum albumin, 2 mM EDTA), treated with fragment crystallizable (Fc) Receptor-Block (Miltenyi Biotec, used 1:100) when antibody stained and then re-suspended in the buffer used for washing. Staining for LDLR was performed in 100 μL of MACS buffer, incubating cells with antibody anti-LDLR conjugated with APC (clone 472413, dilution 1:10, R&D Systems) for 20 minutes at 4° C. in the dark.

### In Vitro Experiments

Transduction of cell lines and human primary cells was performed by adding the desired multiplicity of infection (MOI) of LV to previously counted and plated cells. MOI was calculated as number of vector TU per cell.

### PCSK9 Enzyme-Linked Immunosorbent Assay (ELISA)

The concentration of human PCSK9 was determined in LV-containing supernatants by an ELISA specific for human PCSK9 antigen (Human Proprotein Convertase 9/PCSK9 DuoSet ELISA, R&D Systems) following the manufacturer's instructions. Absorbance of each sample was determined spectrophotometrically at 450 nm, using a Multiskan GO microplate reader (Thermo Fisher Scientific) and normalized to standard curves.

### Mouse Experiments

C57BL/6 mice were purchased from Charles River Laboratories. All the mice were maintained in specific pathogen-free conditions. Vector administration was carried out in adult C57BL/6 mice by tail-vein injections of 2.5×10¹⁰ TU/Kg LV dose. Mice were bled from the retro-orbital plexus using capillary tubes and blood was collected into 0.38% sodium citrate buffer, pH 7.4, for plasma preparation. All animal procedures were performed according to protocols approved by the Institutional Animal Care and Use Committee.

### Factor IX ELISA

The concentration of human Factor IX (FIX) was determined in mouse plasma by an ELISA specific for human FIX antigen (Asserachrom IX:Ag, Stago) following the manufacturer's instructions. Mouse plasma samples were diluted 1:50 or 1:100. Absorbance of each sample was determined spectrophotometrically at 450 nm, using a Multiskan GO microplate reader (Thermo Fisher Scientific) and normalized to Ag standard curves.

### Electron Microscopy

A few microliters of concentrated LV batches were absorbed on glow discharged carbon coated formvar copper grids and fixed for 20 minutes with 4% paraformaldehyde in PBS. After several washes in 50 mM glycine in PBS, grids were blocked in 1% BSA in PBS and incubated with primary antibody diluted in blocking buffer for 30-90 minutes (anti-VSV.G, KeraFAST, 1:50). After several washes in 0.1% BSA in PBS, samples were incubated for 30 minutes with Protein A-gold (5 or 10 nm), fixed with 1% glutaraldehyde, stained with 2% uranyl acetate, or a mix of 0.4% uranyl acetate and 1.8% methylcellulose, then air-dried. Grids were observed with a Zeiss LEO 512 transmission electron microscope. Images were acquired by a 2k×2k bottom-mounted slow-scan Proscan camera controlled by EsivisionPro 3.2 software. For quantification of labeling density, random images of viral particles were taken at nominal magnification of 16k, and gold particles associated to virions were manually counted using ImageJ. Virions were defined based on expected size (approximately 120 nm) and an electron-dense core.

### Results

We generated two plasmids encoding for human wild-type or mutated (S127R) PCSK9, in order to obtain overexpression of the protein by adding one of these plasmids to the transfection mix during LV production. We produced LV expressing GFP under the control of the ubiquitous phosphoglycerate kinase (PGK) promoter (PGK.GFP), with or without the addition of PCSK9-encoding plasmids. We collected LV-containing supernatant 54 hours after transfection, one day later compared to the standard protocol, in order to increase productivity and possible reinfection of producer cells. In this setting, we tested if PCSK9 overexpression could result in an increase of the former by reducing the latter. Interestingly, we observed a 1.6 and a 2-fold increase in infectious titer by adding pMAX.coPCSK9 or pMAX.coPCSK9.S127R, respectively, to the transfection mix (FIG. 1A). Physical particles and infectivity were also slightly increased (FIG. 1B, C). We also measured the concentration of PCSK9 protein in the LV-containing medium by ELISA and found a 2.9-fold reduction of the protein when cells were transfected with plasmid encoding for the mutated PCSK9 compared to the wild-type form (FIG. 1D). We then performed flow cytometry analysis of producer cells, after harvesting LV-containing medium, to measure the content of LDLR on their membrane. We detected a clear reduction of LDLR mean of fluorescence intensity (MFI) when cells were transfected with pMAX.coPCSK9.S127R (FIG. 1E), indicating effective inhibition of LDLR recycling on the plasma membrane of producer cells.

Subsequently we decided to test the transduction potential of LV produced in presence of PCSK9 by exploiting clinically relevant models. Firstly, we transduced in vitro primary human T cells from 3 different donors with PGK.GFP LV produced with or without pMAX.coPCSK9.S127R. We observed a 1.5-2-fold increase in VCN of cells transduced with LV produced with PCSK9 compared to cells transduced with control LV (FIG. 2A). We then produced LV encoding for human factor IX (hFIX) under the control of a hepatocyte-specific expression cassette (ET.FIX) in presence or absence of PCSK9-encoding plasmids and administered them i.v. to adult C57BL/6 mice. We collected plasma of mice to quantify the amount of hFIX over time and 12 weeks after LV administration we harvested the livers to measure VCN. Blood concentrations of hFIX and VCN of mice treated with LV produced with pMAX.coPCSK9.S127R were 1.5-2-fold higher compared to those of mice treated with control LV (FIG. 2B, C). Mice treated with LV produced with pMAX.coPCSK9 showed comparable hFIX amount and VCN to control mice. Overall, these data suggest that overexpression of wild-type PCSK9 does not negatively impact transduction efficiency of LV, while LV produced in presence of PCSK9.S127R may be even more efficient than control LV in transducing human T cells, ex vivo and the mouse liver, in vivo. We hypothesized that during LV production LDLR binds VSV.G also intracellularly, causing VSV.G sequestration and thus lowering the content of VSV.G on the LV envelope. In this scenario, overexpression of PCSK9.S127R, which induces the degradation of LDLR, might have an impact also on the availability of VSV.G for incorporation into the LV envelope. Previous work from our group showed that higher VSV.G content on LV particles corresponds to higher transduction efficiency, especially in T cells and hematopoietic stem and progenitor cells. Thus, we quantified by immune staining and electron microscopy the amount of VSV.G on the envelope of LV particles produced with or without pMAX.coPCSK9.S127R (FIG. 3A, B). Strikingly, we observed an increase of 1.5-fold in VSV.G amount on the envelope of virions produced in the presence of PCSK9 compared to control virions (FIG. 3C). These data show that overexpression of the LDLR inhibitor PCSK9.S127R during LV production increase LV infectious titer of about 2-fold and leads to production of improved LV, with increased VSV.G surface content and higher transduction efficiency of clinically relevant cell types, such as T cells.

Various features and embodiments of the present invention will now be described with reference to the following numbered paragraphs (paras):

1. An enveloped viral particle producer or packaging cell, wherein the cell is modified to decrease expression of low density lipoprotein receptor (LDLR) on the surface of the cell.
2. The cell of para 1, wherein the cell comprises a heterologous polynucleotide comprising a nucleotide sequence encoding pro-protein convertase subtilisin/Kexin type 9 (PCSK9).
3. A method of producing enveloped viral particles comprising the steps:
(a) introducing a transfer vector and optionally one or more helper vector into a cell;
(b) introducing a vector comprising a nucleotide sequence encoding pro-protein convertase subtilisin/Kexin type 9 (PCSK9) into the cell;
(c) culturing the cell under conditions suitable for the production of the enveloped viral particles.
   4. The cell or method of para 2 or 3, wherein the nucleotide sequence encoding PCSK9:
(a) comprises or consists of a nucleotide sequence that has at least 75% sequence identity to SEQ ID NO: 1 or 2; or
(b) comprises or consists of the nucleotide sequence of SEQ ID NO: 1 or 2.
   5. The cell or method of any one of paras 2-4, wherein the PCSK9:
(a) comprises or consists of an amino acid sequence that has at least 75% sequence identity to SEQ ID NO: 3 or 4; or
(b) comprises or consists of the amino acid sequence of SEQ ID NO: 3 or 4.
   6. The cell or method of any one of paras 2-5, wherein the PCSK9 comprises the mutation S127R, wherein the amino acids are numbered with reference to SEQ ID NO: 4.
   7. The cell or method of any preceding para, wherein the cell is a HEK-293 cell or a derivative thereof.
   8. The cell or method of any preceding para, wherein the enveloped viral particle is a lentivirus.
   9. The cell or method of any preceding para, wherein the enveloped viral particle is vesicular stomatitis virus glycoprotein G (VSV-G) pseudotyped.
   10. Use of the enveloped viral particle producer cell according to any one of paras 1-2 or 4-9 for the production of enveloped viral particles.
   11. A method of producing enveloped viral particles comprising the steps of:
(a) providing an enveloped viral particle producer cell according to any one of paras 1-2 or 4-9; and
(b) culturing the cell under conditions suitable for the production of enveloped viral particles.
   12. An enveloped viral particle produced by the method of any one of paras 3-9 or 11.
   13. A cell transduced by the enveloped viral particle of para 12.
   14. The enveloped viral particle or cell of para 12 or 13 for use in therapy.
   15. The enveloped viral particle for use according to para 14, wherein the therapy comprises administering the enveloped viral particle to a subject in need thereof.

All publications mentioned in the above specification are herein incorporated by reference. Various modifications and variations of the disclosed viral particle, cell, method or use of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. An enveloped viral particle producer or packaging cell, wherein the cell is modified to decrease expression of low density lipoprotein receptor (LDLR) on the surface of the cell.

2. The cell of claim 1, wherein the cell comprises a heterologous polynucleotide comprising a nucleotide sequence encoding pro-protein convertase subtilisin/Kexin type 9 (PCSK9).

3. A method of producing enveloped viral particles comprising the steps:
(a) introducing a transfer vector and optionally one or more helper vector into a cell;
(b) introducing a vector comprising a nucleotide sequence encoding pro-protein convertase subtilisin/Kexin type 9 (PCSK9) into the cell;
(c) culturing the cell under conditions suitable for the production of the enveloped viral particles.

4. A method of producing enveloped virus-like particles (VLPs) comprising the steps:
(a) introducing one or more helper vector into a cell;
(b) introducing a vector comprising a nucleotide sequence encoding pro-protein convertase subtilisin/Kexin type 9 (PCSK9) into the cell; and
(b) culturing the cell under conditions suitable for the production of the enveloped VLPs.

5. The cell of claim 2, wherein the nucleotide sequence encoding PCSK9:
(a) comprises or consists of a nucleotide sequence that has at least 75% sequence identity to SEQ ID NO: 1 or 2; or
(b) comprises or consists of the nucleotide sequence of SEQ ID NO: 1 or 2.

6. The cell of claim 2, wherein the PCSK9:
(a) comprises or consists of an amino acid sequence that has at least 75% sequence identity to SEQ ID NO: 3 or 4; or
(b) comprises or consists of the amino acid sequence of SEQ ID NO: 3 or 4.

7. The cell of claim 2, wherein the PCSK9 comprises the mutation S127R, wherein the amino acids are numbered with reference to SEQ ID NO: 4.

8. The cell of claim 1, wherein the cell is a HEK-293 cell or a derivative thereof.

9. The cell of claim 1, wherein the enveloped viral particle is a lentivirus or wherein the enveloped VLP is a lentivirus VLP.

10. The cell of claim 1, wherein the enveloped viral particle or VLP is vesicular stomatitis virus glycoprotein G (VSV-G) pseudotyped.

11. A method of producing enveloped viral particles comprising using the enveloped viral particle producer cell according to claim 1.

12. A method of producing enveloped virus-like particles (VLPs) comprising using the enveloped viral particle packaging cell according to claim 1.

13. A method of producing enveloped viral particles comprising the steps of:
(a) providing an enveloped viral particle producer cell according to claim 1; and
(b) culturing the cell under conditions suitable for the production of enveloped viral particles.

14. A method of producing enveloped virus-like particles (VLPs) comprising the steps of:
(a) providing an enveloped viral particle packaging cell according to claim 1; and
(b) culturing the cell under conditions suitable for the production of enveloped VLPs.

15. An enveloped viral particle produced by the method of claim 3.

16. An enveloped virus-like particle (VLP) produced by the method of any claim 4.

17. A cell transduced by the enveloped viral particle of claim 15.

18. A method of treating a disease or a condition comprising administering the enveloped viral particle claim 15 to a subject in need thereof.

19. A method of treating a disease or a condition comprising administering the enveloped VLP according to claim 16 to a subject in need thereof.

20. A cell transduced by the enveloped VLP of claim 16.
